(19) United States
(12) Patent Application Publication
(10) Pub. No.: US 2024/0269353 A1
(43) Pub. Date: Aug. 15, 2024

(54) NITRIC OXIDE-RELEASING DEVICES

(71) Applicant: Know Bio, LLC, Durham, NC (US)

(72) Inventors: Mark Tapsak, Durham, NC (US); Alice Widman, Duham, NC (US); Andrew P. Gaudet De Lestard, Durham, NC (US)

(21) Appl. No.: 18/562,945

(22) PCT Filed: May 23, 2022
(86) PCT No.: PCT/US22/30538

§ 371 Date: Nov. 21, 2023

Related U.S. Application Data
(60) Provisional application No. 63/191,726, filed on May 21, 2021.
(60) Provisional application No. 63/191,773, filed on May 21, 2021.

(51) Int. Cl. A61L 31/16; A61B 5/145; A61L 27/34; A61L 27/54; A61L 27/58; A61L 31/10; A61L 31/14

(52) U.S. Cl. CPC A61L 31/16; A61B 5/14532; A61L 27/34; A61L 27/54; A61L 27/58; A61L 31/10; A61L 31/148; A61L 2300/114; A61L 2420/04

(57) ABSTRACT

Nitric oxide releasing particles, coatings, tapes, monoliths, and sprayable formulations for reducing the normal foreign body response (FBR) to implanted materials, enhancing wound healing, and/or increasing vascularization. The particles, coatings, tapes, monoliths, and sprayable formulations comprise a biodegradable polymer and an NO-releasing donor compound, and/or are formed of a biodegradable polymer with pendant NO-releasing functional groups.

### CROSS-REFERENCE TO RELATED APPLICATIONS

The present patent application claims the benefit and priority of U.S. Provisional Patent Application No. 63/191,726 filed on May 21, 2021, titled “Nitric Oxide-Releasing Implanted Devices,” and U.S. Provisional Patent Application No. 63/191,773 filed on May 21, 2021, titled “Extending NO Release from Medical Devices Using Donor Embedded Biodegradable Particles,” the contents of which is hereby incorporated by reference in its entirety.

### FIELD

The present disclosure is directed to the use of polymeric coatings, tape, monoliths or sprays to prevent, treat, or minimize the impact of foreign body response, particularly with respect to percutaneous and/or subcutaneous implants, such as continuous glucose monitors. Medical devices that include polymeric coatings that release nitric oxide over time, and implanted medical devices that include an adhered polymeric tape or monolith that releases nitric oxide over time, or which have been sprayed with a polymeric solution that releases nitric oxide over time, are also disclosed. In some embodiments, the coatings include polymers that release nitric oxide, and in other embodiments, the coatings, tapes, monoliths, or sprays include embedded particles, where the particles comprise biodegradable polymers that release nitric oxide, and/or comprise polymers, such as biodegradable polymers, that release nitric oxide or encapsulate compounds, including small molecules, that release nitric oxide.

### BACKGROUND

There are many types of medical devices that are implanted into a human or animal patient, including percutaneous and subcutaneous implants.

Under ideal conditions, implants should initiate the desired host response, and not cause any undesired reaction from neighboring or distant tissues. However, the interaction between the implant and the tissue surrounding the implant can lead to complications, including infection, inflammation, and pain, as well as rejection due to implant-induced coagulation, and allergic foreign body response.

One example of a percutaneous implant is a glucose monitor. Glucose monitors are percutaneously implanted, but are only accurate for a limited time period, due to the host's immune response, called the foreign body response (FBR), to an implanted foreign object.

The FBR initiates upon the insertion of almost any material into subcutaneous tissue, starting with the creation of a wound and the wound healing cascade. Instantaneously, proteins adhere to the biomaterial surface in a process referred to as biofouling. The initial protein adsorption is an integral part of the overall FBR as the ensuing interface promotes the adhesion of inflammatory cells that subsequently stimulate blood clotting and the development of a provisional matrix.

As part of the FBR, macrophages, monocytes, mast cells, and fibroblasts are recruited to the implant site to initiate clearance of the foreign body by releasing chemokines and cytokines. The concentrations and types of mediators released elicit further cell recruitment and ultimately phagocytosis as the body attempts to digest the implant.

This process can result in a local pH's dropping as low as 3.6 and disrupting biosensor performance, as the activity of GOx is pH dependent. While preventing all macrophage migration and subsequent phagocytosis at a wound (glucose sensor) is unlikely, the activation state (i.e., M1 or M2) of the macrophage may influence the overall FBR. Indeed, macrophages serve three primary functions in the body: host defense, wound healing, and immune regulation.

As the FBR progresses, frustrated phagocytosis from activated macrophages will lead to the fusion of macrophages into foreign body giant cells (FBGCs) that attempt to further breakdown the implant. For example, FBGC formation on polyurethane coatings over the biosensors has been shown to promote cracking of the underlying biomaterial.

After one to two weeks, inflammatory cells deposit a collagen matrix that sequesters the implanted device from the native tissue. This collagen encapsulation lacks the microvasculature of native tissue.

When glucose sensors become encapsulated, it hinders accurate measurement of blood glucose. The extent of capsule development is dependent on all other preceding components and rates of progression of the FBR, including protein adhesion, cell activation, and cytokine signaling. The collagen encapsulation will persist for the lifetime of the device, negatively impacting sensor performance with respect to sensitivity and response (e.g., lag times).

Following protein adhesion/biofouling, the FBR proceeds with inflammatory cells responding to the injury, initiating a more profound immune response to the device. For example, mast cells, regulators of inflammation, can also have activity on the implanted biosensors. This has been verified by implanting materials in mast cell-sufficient and-deficient mice, where mast cell-deficient mice implanted with subcutaneous glucose sensors had markedly superior sensor performance than mast cell-sufficient mice. Mast cell-deficient mice exhibited reduced fibrosis and inflammation at the implantation site. One approach for addressing FBR, therefore, has been to use “antifouling materials,” such as polyurethane-coated glucose biosensors.

The most characteristic outcome of the FBR is collagen encapsulation around the foreign device. Early investigations of capsules formed around sensors focused on the influence of the capsule on glucose diffusion from native tissue. Glucose sensitivity correlates with collagen encapsulation, with thicker collagen resulting in greater sensitivity loss.

Increases in mass transfer increase lag times, and can potentially decrease the magnitude and differences when fluctuating between high and low glucose sensor signals. Increases to mass transfer potentially originate from collagen capsule thickness, blood vessel density, or other unanticipated factors associated with FBR.

There are a number of FBR effects on glucose sensor performance, including angiogenesis, cellular glucose consumption, capsule thickness, capsule diffusion coefficient, and capsule porosity. Mathematical modeling suggests that collagen capsule thickness is an important source of sensor lag time with little impact on sensor response attenuation, and sensor attenuation can be reduced with by decreasing the capsule density and increasing angiogenesis around the implanted biosensor. For this reason, biosensors are only accurate for around two weeks before they need to be replaced. The short period of time that they can be used, and the instability of the injected biosensors, has led to poor patient compliance.

As the events in the FBR directly impact the utility of CGM devices, significant research has focused on improving the biocompatibility of these devices as a strategy to improve sensor performance. These strategies range from chemical alteration at the tissue-sensor interface, changing physical properties of the device, and the release of biologically active molecules to influence the tissue reaction. Examples include vascular endothelial growth factor (VEGF), as well as compounds that release nitric oxide (see, for example, Nichols, Scott P et al. “The effect of nitric oxide surface flux on the foreign body response to subcutaneous implants.” Biomaterials vol. 33, 27 (2012)).

Nichols disclosed that the release of nitric oxide (NO) from biomaterials reduces the foreign body response (FBR), though the optimal NO release kinetics and doses remained unknown. Nichols evaluated polyurethane-coated wire substrates with varying NO release properties, which were implanted into porcine subcutaneous tissue. Histological analysis revealed that materials with short NO release durations (i.e., 24 h) were insufficient to reduce the collagen capsule thickness at 3 and 6 weeks, whereas implants with longer release durations (i.e., 3 and 14 d) and greater NO payloads significantly reduced the collagen encapsulation at both 3 and 6 weeks. The acute inflammatory response was mitigated most notably by systems with the longest duration and greatest dose of NO release, supporting the notion that these properties are most critical in circumventing the FBR for subcutaneous biomedical applications (e.g., glucose sensors).

A limitation of the NO-releasing coatings is that the “payload” is limited, and once all the available NO has been released, there is no effective way to produce additional NO to inhibit the foreign body response. Another limitation is that the half-life of nitric oxide release is often too low to delay the onset of the foreign body response.

One attempt to increase the half-life of nitric oxide release has been to include S-nitrosothiol-modified, semi-porous silica particles capable of nitric oxide (NO) release in polyurethane coatings. Such particles are disclosed, for example, in Riccio et al., Chem. Mater. 2011, 23, 7, 1727-1735 (Mar. 7, 2011), where thiol precursors were modified to form S-nitrosothiol NO-releasing functional groups, and introduced into the silica network via co-condensation of mercaptosilane and alkoxysilane precursors. Behaving similarly to low molecular weight S-nitrosothiol containing NO donor compounds, the NO release from the macromolecular silica vehicles was influenced by light, temperature, moisture, and metal ions. Mark Schoenfisch pioneered the use of mesoporous silica nanoparticle (MSNs), and demonstrated that the pores within which the donor moiety can be attached provide a protective environment for the NO payload.

While the use of such MSNs can improve the release kinetics, i.e., extend the time for release as compared to placing the donor moiety in the surface of a silica nanoparticle, a disadvantage to this approach is that the silica particles must be meticulously immobilized within the medical device. It is undesirable to have the particles break away from the device and remain in the host after the device is removed, since these particles are not biodegradable, and are thus persistent in the body. Accordingly, when these particles are included in polymeric coatings that overlie glucose sensors, it is important that the particles not migrate from the coatings.

As with percutaneous implants, there are many issues associated with the surgical implantation of subcutaneous implants, such as artificial joints, pacemakers, and the like. When these medical devices are implanted, and for days, or even weeks later, there are a number of potential risks. These risks include infection, poor wound healing, poor blood supply around the implanted device, and scarring, any of which can damage the patient. Nitric oxide inhibits microbes, such as bacteria, viruses, and fungi, increases vasculature, promotes wound healing, and decreases scarring. However, many NO-releasing functional groups release nitric oxide over relatively short periods, and are not suitable for preventing these types of injuries. It would be advantageous to provide coatings for these materials that release nitric oxide over a sufficiently long period of time that they can help minimize the problems associated with surgical implantation of subcutaneous implants.

When the site of a subcutaneous implant becomes infected during or after surgery, the surrounding tissue becomes infected by microorganisms. Three main categories of infection can occur after operation. Superficial immediate infections are caused by organisms that commonly grow near or on skin. The infection usually occurs at the surgical opening. Deep immediate infection, the second type, occurs immediately after surgery at the site of the implant. Skin-dwelling and airborne bacteria cause deep immediate infection. These bacteria enter the body by attaching to the implant's surface prior to implantation. Though not common, deep immediate infections can also occur from dormant bacteria from previous infections of the tissue at the implantation site that have been activated from being disturbed during the surgery. The last type, late infection, occurs months to years after the implantation of the implant. Late infections are caused by dormant, blood-borne bacteria attached to the implant prior to implantation. The blood-borne bacteria colonize on the implant and, eventually, are released from it. Infusion of the implant with antibiotics can lower the risk of infections during surgery, but only certain types of materials can be infused with antibiotics, and the use of antibiotic-infused implants runs the risk of patient rejection since the patient may develop a sensitivity to the antibiotic, and not every antibiotic works on every type of bacteria.

Inflammation is a common occurrence after any surgical procedure, and is the body's response to tissue damage as a result of trauma, infection, intrusion of foreign materials, or local cell death, or as a part of an immune response.

Implant-induced coagulation is similar to the coagulation process done within the body to prevent blood loss from damaged blood vessels. However, coagulation processes are triggered from proteins that become attached to the implant surface and lose their shapes. When this occurs, the protein changes conformation, and different activation sites become exposed, which may trigger an immune system response where the body attempts to attack the implant to remove the foreign material.

The trigger of the immune system response can be accompanied by inflammation, which may lead to chronic inflammation, in which case, an implant may be rejected and need to be removed from the patient.

The immune system may encapsulate the implant as an attempt to remove the foreign material from the site of the tissue by encapsulating the implant in fibrinogen and platelets. The encapsulation of the implant can lead to further complications, since the thick layers of fibrous encapsulation may prevent the implant from performing the desired functions.

Bacteria may attack the fibrous encapsulation and become embedded into the fibers. Since the layers of fibers are thick, antibiotics may not be able to reach the bacteria, and the bacteria may grow and infect the surrounding tissue. In some cases, it is necessary to remove the implant to remove the bacteria.

Additionally, the body may initiate an allergic foreign body response, which, if several, may result in the implant needing to be removed.

One way to mitigate these adverse events is to expose the area surrounding the implant to nitric oxide. Nitric oxide is known to reduce the foreign body response (FBR). Further, nitric oxide is anti-inflammatory, and can minimize platelet aggregation, thus minimizing implant-induced coagulation. Further, nitric oxide is effective at treating a wide variety of bacterial infections, so can be useful against many of the bacteria that may be introduced to the implant, and may be effective in treating superficial immediate infections, deep immediate infections, and late infections, particularly if nitric oxide release occurs over a period of time of at least two weeks following implantation.

The use of nitric oxide-releasing coatings on implants results in many of the same limitations observed with nitric oxide release on percutaneous implants. With respect to the foreign body response, materials with short NO release durations (i.e., 24 hour) are typically insufficient to reduce the collagen capsule thickness at 3 and 6 weeks, whereas implants with relatively longer release durations (i.e., 3 and 14 days) and greater NO payloads significantly reduce the collagen encapsulation at both 3 and 6 weeks. The acute inflammatory response can be mitigated by systems with the longest duration and greatest dose of NO release, supporting the notion that these properties are most critical in circumventing the FBR for subcutaneous biomedical applications.

One attempt to protect the NO payload and thereby facilitate long-term release has been to embed a crystalline small molecule donor compound within a polymer film. Mark E. Meyerhoff et al, have demonstrated that a polyurethane film doped with S-nitroso-N-acetyl-D-penicillamine can release levels of NO, similar to that of endogenous amounts, for more than 20 days (see, for example, Brisbois, Handa, Major, Bartlett, and Meyerhoff, “Long-term nitric oxide release and elevated temperature stability with S-nitroso-N-acetylpenicillamine (SNAP)-doped Elast-eon E2As polymer. Biomaterials,” 34: 6957-66 (2013)). In addition, Hopkins et al. (Hopkins, et al, “Achieving Long-Term Biocompatible Silicone via Covalently Immobilized S-Nitroso-N-acetylpenicillamine (SNAP) That Exhibits 4 Months of Sustained Nitric Oxide Release,” ACS Applied Materials & Interfaces, Vol. 10, 10.1021/acsami.8b08647 (2018)) has reported a covalently immobilized S-nitroso-N-acetylpenicillamine within silicone rubber that exhibits 4 months of sustained NO release. Both of these examples support the notion that the environment within which the NO donor compound resides is influential in the stability of the compound. The first by Meyerhoff, is a hydrophobic polyurethane, the second by Hopkins is a hydrophobic silicone. Arguably, the disadvantage of these concepts is that they cannot be applied to those systems included above that require a more hydrophilic material/coating/bulk component.

Accordingly, it can be advantageous to provide implants with the ability to release nitric oxide, particularly if the release can occur for relatively longer release durations. However, one limitation associated with current medical devices, including subcutaneous implants, but also including certain percutaneous implants, is that it may require a significant amount of regulatory approval to modify an existing implant to include an NO-releasing coating, so device manufacturers may not wish to modify existing devices such that they release nitric oxide.

It would be advantageous to provide subcutaneous and/or percutaneous implants that release nitric oxide, ideally without any additional regulatory hurdles associated with adding a coating to the implants. The present disclosure provides such implants.

It would also be advantageous to provide additional methods for inhibiting the foreign body response to percutaneous glucose biosensors, so that the sensors maintain their accuracy for relatively longer periods than conventional percutaneous glucose biosensors, while avoiding the limitations associated with using non-biodegradable materials. It would also be advantageous to have ways for applying coatings to other percutaneous and subcutaneous implants that help minimize the impact of the foreign body response. The present disclosure provides such methods, as well as devices to implement these methods.

### SUMMARY

In one embodiment, implants, such as percutaneous implants, comprising a coating that includes biodegradable polymers are disclosed. In one aspect of this embodiment, the implant is a percutaneous continuous glucose monitor.

In various aspects of this embodiment, the coating can be formed of a biodegradable, biocompatible polymer that comprises pendant nitrosothiol (SNO) groups. Following implantation, nitrosothiol groups on the polymer surface are exposed to biological fluids, which cause the nitrosothiol groups to release nitric oxide. The biodegradable polymer can be hydrophilic or hydrophobic, but in order to delay degradation, and extend the release of nitric oxide, it can be preferred that the polymer be hydrophobic.

In other aspects of this embodiment, the coating may or may not include nitrosothiol groups, but comprises embedded particles, such as micro or nano-particles, which are prepared from small molecules and/or polymeric compounds which comprise nitrosothiol groups. The biodegradable polymers may be hydrophilic or hydrophobic, but when introduced into physiological environments where they are exposed to hydrophilic biological fluids, the polymers are preferably hydrophobic, to delay the release of nitric oxide.

In some aspects of these embodiments, the biodegradable polymers comprise monomeric units that are acids, such as lactic acid or glycolic acid, or which are acid anhydrides, so that as the polymer biodegrades, the local pH is acidic. Nitrosothiols tend to release nitric acid faster at relatively acidic pHs, relative to neutral pHs (István Hornyák, Krisztina Marosi, Levente Kiss, Pál Gróf & Zsombor Lacza (2012) Increased stability of S-nitrosothiol solutions via pH modulations, Free Radical Research, 46:2, 214-225), so the presence of relatively low pH (i.e., around 5.5-6.8) in the local environment may promote nitric oxide release.

In another embodiment, subcutaneous implants comprising a coating that includes biocompatible polymers is disclosed. In some embodiments, the polymers are biodegradable, and in other embodiments, they are not biodegradable. In some embodiments, the polymer coating includes embedded particles, which in some aspects of these embodiments, are biodegradable particles.

In some embodiments, a hydrophobic polymer is used for the coating, and hydrophilic or amphiphilic particles are embedded in the polymer coating. In other embodiments, a hydrophobic polymer is used for the coating, and hydrophobic particles are embedded in the polymer coating. In still other embodiments, a hydrophilic or amphiphilic polymer is used for the coating, and hydrophobic particles are embedded in the polymer coating. In still other embodiments, a hydrophilic or amphiphilic polymer is used for the coating, and hydrophilic or amphiphilic particles are embedded in the polymer coating.

The loading of the particles in the polymeric coating can vary, and is typically in the range of between about 1 and about 50% by weight, more typically, between about 5 and about 40% by weight, and preferably between about 10 and about 30% by weight.

Representative subcutaneous implants include artificial joints, pacemakers, stents, insulin infusion sets, shunts, such as hydrocephiletic shunts, reconstructive cosmetic implants, including breast, calf, and butt implants. With respect to stents, the release of nitric oxide from the coatings on the stents can minimize restenosis.

In various aspects of this embodiment, the coating can be formed of a biodegradable, biocompatible polymer that comprises pendant nitrosothiol (SNO) groups. Following implantation, nitrosothiol groups on the polymer surface are exposed to hydrophilic biological fluids, which cause the nitrosothiol groups to release nitric oxide. As the polymer surface biodegrades, a fresh polymer surface is continuously exposed. Depending on the hydrophobicity of the polymer, once the polymer is exposed to moisture, both interior and surface nitrosothiol groups may deliver their NO payload, or the interior nitrosothiol groups may be protected, and the polymer may continuously release nitric oxide as the polymer biodegrades. The more hydrophilic the polymer, the more rapid the release of nitric oxide, as the release may occur, either from the polymer, or from embedded particles within the polymer, soon after the polymer becomes fully hydrated.

Accordingly, in some embodiments, particularly where a hydrophobic polymer is used, nitric oxide is released until the coating completely biodegrades. In other embodiments, particularly where a hydrophilic polymer is used, full release of nitric oxide may occur shortly after a hydrophilic polymer. When the polymer is a hydrophobic polymer, it can provide extended nitric oxide release relative to hydrophilic polymers, whether or not pendant nitrosothiol groups are present on the polymer, or on particles embedded within the polymer.

In some embodiments, the coating is formed of a biodegradable, biocompatible polymer that comprises pendant nitrosothiol (SNO) groups. Following implantation, nitrosothiol groups on the polymer surface are exposed to biological fluids, which cause the nitrosothiol groups to release nitric oxide. As the polymer surface biodegrades, a fresh polymer surface is continuously exposed, which continuously releases nitric oxide. Accordingly, nitric oxide is released until the coating completely biodegrades. In other embodiments, particles comprising nitrosothiol groups are embedded in the polymer coating, and are released as the coating biodegrades. Nitric oxide is then released from the particles as the nitrosothiol groups react in the local environment.

In one embodiment, rather than being an actual coating, the NO-releasing polymers, and/or NO-releasing particles embedded in biodegradable polymers, can be used to form biodegradable sutures, staples, and/or adhesive tapes. These devices can be used, for example, to close a wound or surgical incision, while also releasing nitric oxide over time, which can aid in wound healing, increasing vascularization, minimize scarring, and reduce instances of infection.

In another embodiment, a surgical glue that releases nitric oxide over time is disclosed. In one aspect of this embodiment, the surgical glue comprises a biodegradable polymer with pendant SNO or other NO-releasing groups, which polymer releases nitric oxide over time. Surgical glues comprising biodegradable polymers that include pendant SNO or other NO-releasing groups release nitric oxide over time as the functional groups react, under physiological conditions. In order to include pendant SNO groups, the biodegradable polymer can, for example, be produced from monomers comprising thiol groups, such as thiolactic acid or cysteine, and, optionally, one or more of glycolic acid, lactic acid, and caprolactone. The resulting polymer includes pendant thiol groups that can be converted to SNO groups using known chemistry before the surgical glue is applied. Where the polymer comprises pendant amine groups, the amine groups can be converted to diazeniumdiolate or other suitable NO-releasing functional groups, using known chemistry.

In another aspect of this embodiment, the surgical glue comprises a biodegradable polymer, into which are embedded particles comprising SNO groups or other nitric oxide precursors (NO-releasing functional groups), and, optionally, a biodegradable polymer, which can be a hydrophobic biodegradable polymer.

Surgical glues comprising biodegradable polymers, and embedded particles including SNO or other NO releasing groups, release nitric oxide as the biodegradable polymers degrade over time, releasing the embedded particles, such that the NO-releasing groups in the released particles react, under physiological conditions, to release NO.

In any of these embodiments, the polymer can comprise, in addition to a biodegradable portion, polyethylene glycol branches and/or polymerizable groups, such as (meth)acrylate groups. (Meth)acrylate groups, which, as defined herein, include acrylic acid, methacrylic acid, and C₁₋₆ alkyl esters thereof, can help adhere the surgical glue to a surgical site or a wound site, and polyethylene glycol groups can minimize scarring around an injury/incision. The surgical glues have the added ability of promoting healing by releasing nitric oxide.

In still another embodiment, a biodegradable scaffold for tissue engineering is disclosed. In one aspect of this embodiment, the scaffold is formed from biodegradable polymers that include pendant SNO groups. In another aspect of this embodiment, the scaffold is formed from biodegradable polymers comprising embedded particles, which particles comprise one or more compounds. The biodegradable polymers, and/or embedded particles, can be the same polymers and particles discussed above with respect to the NO-releasing coatings.

In one aspect of this embodiment, the scaffold comprises stem cells, and, optionally various growth factors, which can guide the differentiation of the stem cells into desired cell types. In one aspect of this embodiment, the stem cells proliferate in approximately the same timeframe as the scaffold degrades, thus forming a three-dimensional tissue matrix in approximately the same shape as the scaffold.

In various embodiments, the polymers and/or compounds that include nitrosothiol groups are capable of releasing nitric oxide over an extended period of time, for example, over a week, two weeks, three weeks, or even a month or more.

The degradation time can be controlled by judicious selection of the monomers used to prepare the biodegradable polymers, as well as the percent crystallinity, molecular weight, and hydrophobicity. For example, polyglycolic acid tends to biodegrade faster than polylactic acid, and copolymers of lactic and glycolic acid can be prepared with varying ratios of these monomers, where the degradation time can be controlled.

In any of these embodiments, the biodegradable polymer can be a branch, comb, or graft copolymer.

Included in some embodiments, hydrophobic particles are embedded within hydrophilic coatings. In other embodiments, hydrophilic particles are embedded within hydrophobic coatings. In still other embodiments, hydrophilic particles are embedded within hydrophilic coatings, or hydrophobic particles are embedded within hydrophobic coatings.

The biodegradable polymers can be, for example, branched, comb, or graft copolymers, terpolymers, and the like. Representative monomers used to prepare the polymers include, but are not limited to, saccharides, amino acids, hydroxy acids, such as glycolic, lactic acid, and hydroxybutyric acid, lactones such as caprolactone, carbonates, amino acids and saccharides.

Saccharides form polysaccharides by forming glycosidic bonds, which take a hemiacetal of a saccharide and binds it to an alcohol via loss of water. Amino acids can form peptides and proteins, and when the amino acids include cysteine, the resulting peptide or protein includes pendant thiol groups that can be converted to nitrosothiol groups. Examples of monomers that can be used to form biodegradable polyhydroxycarboxylic acids (a subset of polyesters) include hydroxybutyric acid, glycolic acid, lactic acid, thiolactic acid, and co- and ter-polymers thereof.

Methods of treatment using the devices described herein are also disclosed. For example, methods of promoting wound healing by applying a surgical adhesive that releases nitric oxide are disclosed. Methods of monitoring glucose levels using a percutaneous glucose monitor, with a sensor coated with an NO-releasing coating, are also disclosed. Methods of minimizing foreign body response to subcutaneous implants, by coating the implants with a coating described herein, are also disclosed.

In yet another embodiment, implants, such as subcutaneous implants, and, in some aspects of this embodiment, embodiments, percutaneous implants, comprising an adhered tape or monolith, wherein the tape or monolith releases nitric oxide upon exposure to physiological fluids, are disclosed.

In a further embodiment, implants, such as subcutaneous implants, and, in some aspects of this embodiment, embodiments, percutaneous implants, sprayed with a polymer solution that releases nitric oxide upon exposure to physiological fluids, are disclosed.

In one aspect of these embodiments, the tape, monolith or sprayable formulation comprises biodegradable polymers.

In one aspect of these embodiments, the implant is a sensory implant, neurological implant, cardiac implant, orthopedic implant, electrical implant, contraceptive implant, or cosmetic implant. In some embodiments, all or a portion of the implant is porous.

Representative subcutaneous implants include artificial joints, pacemakers, stents, insulin infusion sets, ports, shunts, such as hydrocephiletic shunts, reconstructive cosmetic implants, including breast, calf, and butt implants. With respect to stents, the release of nitric oxide from the coatings on the stents can minimize restenosis.

In various aspects of this embodiment, the tape, monolith, or sprayable polymer solution can be formed of a biodegradable, biocompatible polymer that comprises pendant NO-releasing functional groups, such as nitrosothiol (SNO) or diazeniumdiolate groups. Following implantation, these NO-releasing functional groups on the polymer surface are exposed to biological fluids, which cause the nitrosothiol groups to release nitric oxide. The biodegradable polymer can be hydrophilic or hydrophobic, but in order to delay degradation, and extend the release of nitric oxide, it can be preferred that the polymer be hydrophobic.

In other aspects of this embodiment, the polymer used in the tape, monolith, or sprayable polymer solution may or may not include NO-releasing functional groups, such as nitrosothiol or diazeniumdiolate groups, but comprises embedded particles, such as micro or nano-particles, which are prepared from small molecules and/or polymeric compounds that comprise these groups. Where biodegradable polymers are used, they may be hydrophilic or hydrophobic, but when introduced into physiological environments where they are exposed to hydrophilic biological fluids, the polymers are preferably hydrophobic, to delay the release of nitric oxide.

In some aspects of these embodiments, the biodegradable polymers comprise monomeric units that are acids, such as lactic acid or glycolic acid, or which are acid anhydrides, so that as the polymer biodegrades, the local pH is acidic. Nitrosothiols tend to release nitric acid faster at relatively acidic pHs, relative to neutral pHs, so the presence of relatively low pH (i.e., around 5.5-6.8) in the local environment may promote nitric oxide release.

In some embodiments, the tape, monolith, or sprayable polymer solution comprises a hydrophobic polymer, and hydrophilic or amphiphilic particles are either embedded in the polymer, in the case of the tape or monolith, or are included in the polymer solution, in the case of a sprayable coating applied to the medical devices.

In other embodiments, the tape, monolith, or sprayable polymer solution comprises a hydrophobic polymer, and hydrophobic particles are either embedded in the polymer, in the case of the tape or monolith, or are included in the polymer solution, in the case of a sprayable coating applied to the medical devices.

In still other embodiments, the tape, monolith, or sprayable polymer solution comprises a hydrophilic or amphiphilic polymer, and hydrophobic particles are either embedded in the polymer, in the case of the tape or monolith, or are included in the polymer solution, in the case of a sprayable coating applied to the medical devices.

In other embodiments, the tape, monolith, or sprayable polymer solution comprises a hydrophilic or amphiphilic polymer, and hydrophilic or amphiphilic particles are either embedded in the polymer, in the case of the tape or monolith, or are included in the polymer solution, in the case of a sprayable coating applied to the medical devices.

The loading of the particles in the polymeric tape, monolith, or sprayable polymer solution can vary, and is typically in the range of between about 1 and about 50% by weight, more typically, between about 5 and about 40% by weight, and preferably between about 10 and about 30% by weight.

In various aspects of these embodiments, the tape, monolith or polymer present in the sprayable formulation comprises a biodegradable, biocompatible polymer that comprises pendant nitrosothiol (SNO) groups. Following implantation, nitrosothiol groups on the polymer surface are exposed to hydrophilic biological fluids, which cause the nitrosothiol groups to release nitric oxide. As the polymer surface biodegrades, a fresh polymer surface is continuously exposed. Depending on the hydrophobicity of the polymer, once the polymer is exposed to moisture, both interior and surface nitrosothiol groups may deliver their NO payload, or the interior nitrosothiol groups may be protected, and the polymer may continuously release nitric oxide as the polymer biodegrades. The more hydrophilic the polymer, the more rapid the release of nitric oxide, as the release may occur, either from the polymer, or from embedded particles within the polymer, soon after the polymer becomes fully hydrated.

Accordingly, in some embodiments, particularly where a hydrophobic polymer is used, nitric oxide is released until the tape, monolith, or sprayed-on coating completely biodegrades. In other embodiments, particularly where a hydrophilic polymer is used, full release of nitric oxide may occur shortly after implantation. When the polymer is a hydrophobic polymer, it can provide extended nitric oxide release relative to hydrophilic polymers, whether or not pendant nitrosothiol groups are present on the polymer, or on particles embedded within the polymer.

In some embodiments, the tape, monolith, or polymeric spray formulation comprises a biodegradable, biocompatible polymer that comprises pendant nitrosothiol (SNO) groups. Following implantation, nitrosothiol groups on the polymer surface are exposed to biological fluids, which cause the nitrosothiol groups to release nitric oxide. As the polymer surface biodegrades, a fresh polymer surface is continuously exposed, which continuously releases nitric oxide. Accordingly, nitric oxide is released until the coating completely biodegrades. In other embodiments, particles comprising nitrosothiol groups are embedded in the tape, monolith, or sprayable polymer solution, and are released as the tape, monolith, or sprayed-on coating biodegrades. Nitric oxide is then released from the particles as the nitrosothiol groups react in the local environment.

In one embodiment, the tapes, monoliths, or sprayable formulations are applied to medical implants prior to implantation, for example, within hours of implantation, rather than a pre-formed coating, which might be applied long before the implant is implanted. This allows the implanted medical devices to release nitric oxide over time, which can aid in wound healing, increasing vascularization, minimize scarring, and reduce instances of infection.

In another embodiment, the sprayable formulation is used in a manner similar to a surgical glue, but instead of being applied to a wound site, it is applied to the implant surface (and optionally into the pores on the surface of porous implants) rather than a wound. However, in some embodiments, the sprayable formulation can be used as a surgical glue, depending on whether or not the sprayable formulation comprises crosslinkable groups that can crosslink on the wound surface, and thus assist in closing the wound.

In some embodiments, where the biodegradable polymer in the tape, monolith or sprayable formulation comprises pendant SNO groups, the biodegradable polymer can, for example, be produced from monomers comprising thiol groups, such as thiolactic acid or cysteine, and, optionally, one or more of glycolic acid, lactic acid, and caprolactone. The resulting polymer includes pendant thiol groups that can be converted to SNO groups using known chemistry before the tape, monolith, or sprayable formulation is applied to the implant. Where the polymer comprises pendant amine groups, the amine groups can be converted to diazeniumdiolate or other suitable NO-releasing functional groups, using known chemistry.

In other embodiments, rather than the biodegradable polymer in the tape, monolith or sprayable formulation comprising pendant SNO groups, particles embedded in the polymer, or mixed in with the polymer solution, comprise SNO groups or other nitric oxide precursors (NO-releasing functional groups), and, optionally, a biodegradable polymer, which can be a hydrophobic biodegradable polymer.

The polymers and/or compounds that include nitrosothiol groups are capable of releasing nitric oxide over an extended period of time, for example, over a week, two weeks, three weeks, or even a month or more.

In any of these embodiments, the polymer can comprise, in addition to a biodegradable portion, polyethylene glycol branches and/or polymerizable groups, such as (meth)acrylate groups. (Meth)acrylate groups, which, as defined herein, include acrylic acid, methacrylic acid, and C₁₋₆ alkyl esters thereof, can help adhere the tape, monolith, and/or sprayable formulation to an implant, and polyethylene glycol groups can minimize scarring around the implant site.

The degradation time can be controlled by judicious selection of the monomers used to prepare the biodegradable polymers, as well as the percent crystallinity, molecular weight, and hydrophobicity. For example, polyglycolic acid tends to biodegrade faster than polylactic acid, and copolymers of lactic and glycolic acid can be prepared with varying ratios of these monomers, where the degradation time can be controlled.

In any of these embodiments, the biodegradable polymer can be a branch, comb, or graft copolymer. The biodegradable polymers can be, for example, branched, comb, or graft copolymers, terpolymers, and the like. Representative monomers used to prepare the polymers include, but are not limited to, saccharides, amino acids, hydroxy acids, such as glycolic, lactic acid, and hydroxybutyric acid, lactones such as caprolactone, carbonates, amino acids and saccharides.

Saccharides form polysaccharides by forming glycosidic bonds, which take a hemiacetal of a saccharide and binds it to an alcohol via loss of water. Amino acids can form peptides and proteins, and when the amino acids include cysteine, the resulting peptide or protein includes pendant thiol groups that can be converted to nitrosothiol groups. Examples of monomers that can be used to form biodegradable polyhydroxycarboxylic acids (a subset of polyesters) include hydroxybutyric acid, glycolic acid, lactic acid, thiolactic acid, and co- and ter-polymers thereof.

Methods of treatment using the devices described herein are also disclosed. For example, methods of minimizing foreign body response to implanted medical devices, by applying the tape, monolith, or sprayable formulations described herein onto the implanted medical devices, and allowing the NO-releasing functional groups in the tapes, monoliths, and/or sprayable formulations to release nitric oxide over time, are also disclosed.

The present invention will be better understood with respect to the following Detailed Description.

### BRIEF DESCRIPTION OF THE DRAWINGS

FIG. 1 is a schematic illustration of a polymer coating comprising NO-releasing biodegradable particles, adhered to a medical device, which medical device is implanted subcutaneously within a host.

FIG. 2 is a schematic illustration of a polymer coating comprising NO-releasing biodegradable particles, adhered to a medical device, which medical device is implanted subcutaneously within a host, showing particles that are released from the coating as the polymer degrades.

FIG. 3 is a schematic illustration of a wound gel or surgical glue comprising NO-releasing biodegradable particles, where the glue or gel is placed within a wound site.

FIG. 4 is a schematic illustration of a tissue engineering scaffold formed from a polymer comprising NO-releasing biodegradable particles, where NO can be released to promote tissue ingrowth, increase vascularization, and/or reduce scar tissue formation.

FIG. 5 is a schematic illustration of the foreign body response to an implanted glucose sensor over time.

### DETAILED DESCRIPTION

In one embodiment, devices and methods are disclosed for releasing exogenous NO from implantable materials and devices, such as percutaneous and subcutaneous implants. The release of exogenous NO can improve local healing, and decrease the FBR to the materials and devices following implantation. In certain embodiments of the implantable devices, nitric oxide release occurs over weeks to months, so as to reduce foreign body response to the devices over a relatively long period of time.

In some embodiments, an important factor to the stabilization of the NO to its donor moiety (i.e., an NO-releasing functional group on a polymer or small molecule) is the water content within which it is stored or deployed within a device. It can be advantageous to keep the NO-donor dry, following implantation into a wet environment (i.e., tissue perfused with aqueous biological fluids). If the implant can be maintained relatively dry, for example, by using hydrophobic polymers in the coating, or in particles encapsulated within the coating, or by using hydrophobic polymers in the tape, monolith, or sprayable formulation, or in particles encapsulated within the tape or monolith, or present in the sprayable formulation, the NO remains bound to the donor for a relatively longer period of time than if hydrophilic polymers were used. When NO-releasing compounds are exposed to water (or water vapor), it dramatically increases the rate by which the NO is released. Unfortunately, it is a requirement for some medical devices, such as glucose sensors and wound care glues, to permit a high-water content, at least around that portion of the active glucose sensor where the electrode resides. However, areas above and below that portion of the active glucose sensor can be coated with hydrophobic polymers.

Percutaneous glucose sensors comprise two portions. A first portion is an active sensing region, where an electrode or other sensing portion determines the glucose concentration in the interstitial space of a user's tissues. A second portion operatively connects the first portion of the implantable glucose sensor to that portion of the glucose monitor that overlies the user's skin, and allows the first portion of the implantable glucose sensor to penetrate the user's skin to a desired depth.

In the case of glucose sensors, the outermost polymer coatings must allow the diffusion of glucose to their sensing regions. The structure of the highly-polar carbohydrate (glucose) requires many water molecules to facilitate this diffusion. For this reason, glucose sensors typically comprise materials that have water contents of 5% by weight or more.

With respect to surgical dressings and wound care glues, water diffusion and solubility within these products is also a requirement for the cellular processes and proliferation that are being promoted to complete the host's healing. Like a glucose sensor, these materials must permit the passage of metabolites such as glucose while at the same time facilitate the diffusion of cellular waste products.

In some embodiments, an important factor to the stabilization of the NO to its donor moiety is the water content within which it is stored or deployed within a device. It can be advantageous to keep the NO-donor dry, following implantation into a wet environment (i.e., tissue perfused with aqueous biological fluids). If the implant can be maintained relatively dry, for example, by using hydrophobic polymers in the tape, monolith, or sprayable formulation, or in particles encapsulated within the tape or monolith, or present in the sprayable formulation, the NO remains bound to the donor for a relatively longer period of time than if hydrophilic polymers were used. When NO-releasing compounds are exposed to water (or water vapor), it dramatically increases the rate by which the NO is released.

The components of, their preparation, and their use in the medical devices described herein are discussed in detail below.

### Polymers

Polymeric materials are commonly used in connection with implanted medical devices, because of the ease of fabrication, flexibility, and their biocompatible nature as well as their wide range of mechanical, electrical, chemical, and thermal behaviors when combined with different materials as composites. Polymeric materials must also have considerable tensile strength and should be able to contain the device over the envisioned lifetime of the implant.

It is intended that the polymers, when adhered to, sprayed onto, or coated onto medical devices, or in some embodiments, used to make the medical devices themselves, comprise NO-releasing functional groups, or include embedded particles or small molecules which include such NO-releasing functional groups. In this section, the polymers are described, and in a later section, methods for functionalizing the polymers, or particles or compounds embedded within the polymers, so that they comprise pendant NO-releasing functional groups, are disclosed.

While it is possible, in some cases, to directly prepare polymers comprising NO-releasing functional groups, these functional groups are relatively unstable to various reaction conditions, particularly those that involve exposing the functional groups to light, heat, moisture, or pH levels below around 7. Accordingly, it is typically easier to first form polymers with pendant functional groups that can be reacted to form NO-releasing functional groups, and then convert the pendant functional groups to NO-releasing functional groups.

As used herein, the term “polymer” has the meaning commonly afforded the term. Examples include homopolymers, co-polymers (including block copolymers and graft copolymers), dendritic polymers, crosslinked polymers and the like. Suitable polymers include synthetic and natural polymers (e.g. polysaccharides, peptides) as well as polymers prepared by condensation, addition and ring opening polymerizations. Also included are rubbers, fibers and plastics. Polymers can be hydrophilic, amphiphilic or hydrophobic. In one aspect, the polymers are non-peptide polymers. In some embodiments, the polymers are biocompatible and/or biodegradable. Certain classes of polymers can be either hydrophilic or hydrophobic, depending on the monomers used to prepare them, the degree of polymerization, and the like. Certain hydrophilic and certain hydrophobic polymers may be biodegradable, and others may not be biodegradable.

A hydrophilic polymer or polymer blend is one in which a film or particle of said polymer will increase in weight by more than 5% when placed into an aqueous solution of phosphate buffered saline (0.9% salinity, pH 7.4) at 37° C. for 24 hours or more.

A hydrophobic polymer or polymer blend is one in which a film or particle of said polymer will not increase in weight by 5% or more when placed into an aqueous solution of phosphate buffered saline (0.9% salinity, pH 7.4) at 37° C. for 24 hours or more.

Biocompatible and biostable polymers are extensively used to package implanted devices, with the main criteria that include gas permeability and water permeability of the packaging polymer to protect the electronic circuit of the device from moisture and ions inside the human body.

Non-degradable polymers are often used where the medical device is an implant that is intended to remain in place for extended periods of time, such as pacemakers, artificial hips, and the like. Representative non-degradable polymers used in connection with implanted medical devices include polyurethane, polyvinylidene fluoride, polyethylene, polypropylene, polydimethylsiloxane, parylene, polyamide, polytetrafluoroethylene, poly(methyl methacrylate), and polyimide, and a number of these polymers are hydrophobic. In total hip arthroplasty, the bearing system typically employs an ultra-high-molecular-weight polyethylene (UHMWPE) insert that articulates against a cobalt-chromium alloy or ceramic in order to restore function to a damaged or diseased joint.

Several polymeric implants and prosthesis are used in clinical practice. One of the most common applications is synthetic menisci. Menisci are cartilage tissues, which serve to disperse friction in the knee joint. Artificial menisci can be prepared using collagen, polyurethane, polyvinyl alcohol, hyaluronic acid, polycaprolactone, and combinations thereof.

A polymer with pendant-S—NO groups is referred to as an S-nitrosated polymer. A polymer with pendant-S—NO₂ groups is referred to as an S-nitrated polymer. An “—S—NO₂ group” is also referred to as a sulfonyl nitrate, an S-nitrothiol or a thionitrate. —SNO and —S—NO₂ groups decompose in vivo, resulting in the delivery of NO. In one aspect, an S-nitrated polymer also has pendant —O—NOX groups. The S-nitrated polymers have at least one NO₂ group per 1200 atomic mass unit of the polymer, preferably, at least one NO₂ group per 600 amu of the polymer, and, even more preferably, at least one NO₂ group per 70 amu of the polymer, with similar concentrations of NO groups on S-nitrosated polymers and on polymers with diazeniumdiolate groups.

### Hydrogels

In some aspects of these embodiments, the polymers are water-insoluble and hydrophilic, and can form hydrogels. A hydrogel is a composition that can absorb large quantities of water. Polymers which can form hydrogels are generally more biocompatible than other polymers and can be used in devices which are inserted into, for example, vascular systems. Hydrogels also generally exhibit extremely mild foreign body reactions during soft tissue implantation.

Polymers that form hydrogels are typically crosslinked hydrophilic polymers. Further descriptions and examples of hydrogels are provided in Hydrogels and Biodegradable Polymers for Bioapplications, editors Attenbrite, Huang and Park, ACS Symposium Series, No. 627 (1996), U.S. Pat. Nos. 5,476,654, 5,498,613 and 5,487,898, the teachings of which are incorporated herein by reference.

Examples of hydrogels include polyethylene glycols, polysaccharides and crosslinked polysaccharides, as well as Eudragit® polymers, which include ethylene glycol and propylene glycol chains.

### Biodegradable Polymers

Biodegradable polymers are polymers which meet the requirements of biocompatibility and biodegrade into harmless end-products. The polymers described below are intended to be modified to include one or more NO-releasing functional groups. In the description of the polymers below, the polymers are disclosed as incorporating functional groups that can be converted to NO-releasing compounds, and elsewhere herein, methods for converting those functional groups to NO-releasing compounds are disclosed.

The biodegradable polymers can be, for example, branched, comb, or graft copolymers, terpolymers, and the like. Representative monomers used to prepare the polymers include, but are not limited to, saccharides, amino acids, hydroxy acids, such as glycolic and lactic acid, hydroxybutyric acid, lactones such as caprolactone. Suitable polymers include polyhydroxyacids, polyanhydrides, polyhydroxyalkanoates, polyesteramides, aliphatic copolyesters, and aromatic copolyesters. Examples of monomers that can be used to form biodegradable polyhydroxyacids (polyesters) include hydroxybutyric acid, glycolic acid, lactic acid, thiolactic acid, and co- and ter-polymers thereof.

One representative class of biodegradable polymer is a linear polyester based on lactic acid, glycolic acid, and mixtures and copolymers thereof (PLGA). PLGA is degraded by ester hydrolysis into lactic acid and glycolic acid, and has been shown to possess excellent biocompatibility. Polycaprolactones and polycarbonates moieties are also biodegradable, and the monomers can be incorporated into PLGA polymers.

Saccharides form polysaccharides by forming glycosidic bonds, which take a hemiacetal of a saccharide and binds it to an alcohol via loss of water. Amino acids can form peptides and proteins, and when the amino acids include cysteine, the resulting peptide or protein includes pendant thiol groups that can be converted to nitrosothiol groups. When the amino acids comprise lysine, the pendant amine groups can be converted to diazeniumdiolates.

In those embodiments where the biodegradable polymers comprise monomeric units that are acids, such as lactic acid or glycolic acid, or which are acid anhydrides, as the polymer biodegrades, the local pH is acidic. NO-releasing groups, such as diazenium diolates and/or nitrosothiols, tend to release nitric acid faster at relatively acidic pHs, relative to neutral pHs, so the presence of relatively low pH (i.e., around 5.5-6.8) in the local environment may promote nitric oxide release.

Thiol groups can be incorporated into the polymers by incorporating a monomer with one or more pendant thiol groups, such as thiolactic acid or cysteine, into the polymerization reaction. The concentration of the thiol-containing monomer can vary depending on the desired amount of NO-release, but is typically in the range of between about 1 and about 50% by weight, more typically, between about 5 and about 25% by weight, and most typically, between about 10 and about 20% by weight.

Where the thiol groups can interfere with the polymerization chemistry, or would be converted to other functional groups, and not be available for later nitrosation to form nitrosothiol groups, the thiol groups can be protected during the polymerization process, and deprotected afterwards. Protecting groups for thiols are well known to those of skill in the art.

PLGA coatings on medical devices degrade through bulk erosion at a uniform rate throughout the matrix. The degradation process is self-catalyzed, as the number of terminal carboxylic acid groups rises with increasing chain scission, and the acids catalyze the hydrolysis. The degradation is highly dependent on the ratio of lactide to glycolide moieties, as lactide is more hydrophobic and reduces the rate of degradation. Also, important factors in the degradation process are the degree of crystallinity, the molecular weight, and the glass transition temperature of the polymer. By controlling the ratio of lactic acid to glycolic acid, and/or incorporating carbonate and/or caprolactone into the polymer backbone, the resulting polymer can be relatively hydrophobic or relatively hydrophilic.

### Hydrophilic Polymers

Hydrophilic polymers have a strong affinity for water. They can be comprised of either synthetic polymers such as polyvinylpyrrolidone and polyethyleneglycol or natural polymers such as proteins and polysaccharides.

Amino acids can form peptides and proteins, and when the amino acids include cysteine, the resulting peptide or protein includes pendant thiol groups that can be converted to nitrosothiol groups.

Polysaccharides are one example of a hydrophilic polymer. Saccharides form polysaccharides by forming glycosidic bonds, which take a hemiacetal of a saccharide and binds it to an alcohol via loss of water.

Representative polysaccharides include cyclodextrins, such as alpha-cyclodextrin, beta-cyclodextrin and gamma-cyclodextrin starches, dextrins, dextrans, ficolls, celluloses, fucoidin, alginic acid, carrageenans, such as K-carrageenan, and glycosaminoglycans, such as hyaluronic acid, chondroitin and glucosamine.

Starches include highly branched starches of relatively low molecular weight (maltodextrin, average molecular weight about 5,000 Da). Starches can be covalently modified with acryl groups for conversion into a form which can be solidified into microspheres, and the polyacryl starch can be converted into particulate form by radical polymerization in an emulsion (see, for example, Characterization of Polyacryl Starch Microparticles as Carriers for Proteins and Drugs, Artursson et al, J Pharm Sci, 73, 1507-1513, 1984)).

Ficoll is a neutral, highly branched, high-mass, hydrophilic polysaccharide which dissolves readily in aqueous solutions. Ficoll is prepared by reacting a polysaccharide with epichlorohydrin.

Dextran is a complex branched glucan (polysaccharide derived from the condensation of glucose). IUPAC defines dextrans as “Branched poly-α-d-glucosides of microbial origin having glycosidic bonds predominantly C-1→C-6”. Dextran chains are of varying lengths (from 3 to 2000 kilodaltons). The polymer main chain consists of α-1,6 glycosidic linkages between glucose monomers, with branches from α-1,3 linkages. This characteristic branching distinguishes a dextran from a dextrin, which is a straight chain glucose polymer tethered by α-1,4 or α-1,6 linkages.

Chitosan is a linear polysaccharide composed of randomly distributed β-(1→4)-linked D-glucosamine (deacetylated unit) and N-acetyl-D-glucosamine (acetylated unit). It is typically made by treating the chitin shells of shrimp and other crustaceans with an alkaline substance, such as sodium hydroxide.

Cellulose is a linear polymer of D-glucose units linked by β(1→4)-glycosidic bonds. Cellulose derivatives can also be used, including cellulose esters and cellulose ethers. Representative cellulose esters include cellulose acetate, cellulose triacetate, cellulose propionate, cellulose acetate propionate (CAP), and cellulose acetate butyrate (CAB). Representative cellulose ethers include carboxymethyl cellulose (CMC), Ethyl hydroxyethyl cellulose, hydroxypropyl methyl cellulose (HPMC), Hydroxyethyl methyl cellulose, hydroxypropyl cellulose (HPC), hydroxyethyl cellulose, ethyl methyl cellulose, ethyl cellulose, and methyl cellulose.

Other suitable examples are disclosed in Bioactive Carbohydrates, Kennedy and White eds., (John Wiley Sons), Chapter 8, pages 142-182, (1983) the teachings of which are incorporated herein by reference. Polysaccharides have pendant primary and secondary alcohol groups. Consequently, S-nitrosylated polysaccharides can be prepared from polythiolated polysaccharides by the methods described herein.

A polysaccharide can be converted to a polythiolated polysaccharide, for example, by the methods disclosed in Gaddell and Defaye and Rojas et al. In these methods, primary alcohols are thiolated preferentially over secondary alcohols. Preferably, a sufficient excess of thiolating reagent is used to form perthiolated polysaccharides. Polysaccharides are “perthiolated” when all of primary alcohols have been converted to thiol groups.

In another aspect, a polythiolated polysaccharide can be prepared by reacting the alcohol groups, preferably the primary alcohol groups, on the polysaccharide with a reagent which adds a moiety containing a free thiol or protected thiol to the alcohol. In one example the polysaccharide is reacted with a bis isocyanatoalkyldisulfide followed by reduction to functionalize the alcohol. Conditions for carrying out this reaction are found in Cellulose and its Derivatives, Fukamota, Yamada and Tonami, eds. (John Wiley & Sons), Chapter 40, (1985) the teachings of which are incorporated herein by reference.

Polysaccharides can also be modified to include one or more thiol-containing sugars, such as the following:

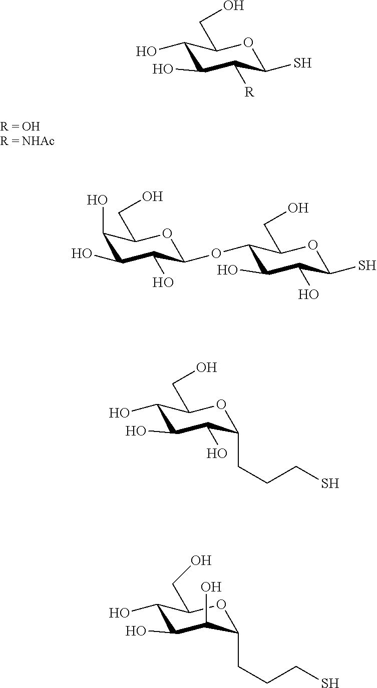

Glucosamine and galactosamine are naturally-occurring amino acid sugars:

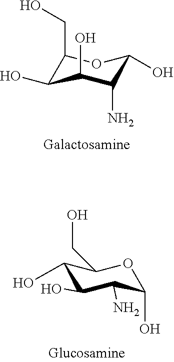

These and other amine-containing sugars can be used to prepare polysaccharides with pendant diazeniumdiolate groups.

### Hydrophobic Polymers

Medical devices coated with hydrophobic biocompatible polymers with pendant NO-releasing groups, or embedded particles or small molecules with such groups, are capable of achieving sustained, local release of nitric oxide. These hydrophobic polymers also can be stable for a certain period and then degrade, to allow the growth of cells/tissues. These biocompatible hydrophobic polymers can be used for drug delivery, tissue augmentation, and regenerative medicine applications.

Synthetic hydrophobic polymers can be subdivided into two groups:

1. Prepolymerized: These polymers are prepolymerized from their monomers, and include, for example, poly(ε-caprolactone) (PCL), poly(lactic acid) (PLA), poly(D,L-lactic-co-glycolic acid) (PLGA), and polystyrene.

2. Polymerized in process: These polymers are synthesized from monomers during the preparation of particles, and include, for example, poly(alkyl cyanoacrylate), poly(isobutyl cyanoacrylate), poly(butylcyanoacrylate), poly(methyl methacrylate), and poly(hexal cyanoacrylate).

Synthetic polymers have the advantage of sustained release over a period of days to several weeks compared to the relatively shorter duration of drug release of natural polymers. Their other benefits includes the use of organic solvents and the requirement of typical conditions during encapsulation. Polymeric NPs have, therefore been widely investigated as drug delivery systems over the past few decades, including the clinical study of Food and Drug Administration (FDA)-approved biodegradable polymeric NPs such as PLA and PLGA

Representative hydrophobic biodegradable injectable polymers include aliphatic polyesters, polycarbonates and polyanhydrides, including those prepared from lactic acid, glycolic acid, caprolactone, aliphatic diols and diacids, hydroxy fatty acids, and triglycerides such as castor oil.

Poly(ortho esters) are highly hydrophobic polymers that contain acid-sensitive links in the polymer backbone. At the physiological pH of 7.4, these links undergo a very slow rate of hydrolysis, but as the ambient pH is lowered, for example, upon exposure to physiological fluids, hydrolysis rates increase. The hydrophobicity of these polymers limits water penetration, thus confining erosion to the surface, leading to a controlled release of nitric oxide.

The degradation rate of hydrophobic polymers can often be controlled, for example, by adjusting the types and ratios of the monomers used to prepare them. For example, PCPP and PCPP-SA 85:15 (poly[bis(p-carboxyphenoxy)propane anhydride] and its copolymer with sebacic acid), have nearly constant erosion kinetics over the course of several months. By altering the CPP/SA ratio, nearly any degradation rate between 1 day and 3 years can be achieved (Leong, K. W., Brott, B. C., and Langer, R., J. Biomed. Mater. Res. (25). Copyright © 1985 John Wiley &amp; Sons. Inc.).

Additional hydrophobic polymers are disclosed, for example, in Rahmani, Mehran, “List of Hydrophobic Polymers and Coating, 10.13140/RG.2.2.26536.72960 (2019).

In some embodiments, the polymers are not brittle, and consequently remain adhered to medical devices, even under physiological conditions. These types of polymers are particularly suited for coating devices which are to be implanted in patients for extended periods of time.

Converting Polymers to Polymers with Pendant NO-Releasing Functional Groups

After the polymers are formed, pendant functional groups on the polymers can be converted to NO-releasing functional groups. This same chemistry can be used to convert pendant functional groups on small molecules to NO-releasing functional groups.

Polymers with pendant NO-releasing functional groups can be prepared from polymers having a multiplicity of nucleophilic functional groups, including amines, thiols, hydroxyls, hydroxylamines, hydrazines, amides, guanadines, imines, aromatic rings and nucleophilic carbon atoms (such as a relatively basic proton alpha to a carbonyl moiety, which, when removed by addition of a base, forms a nucleophilic enolate ion that can react with nitric oxide). However, thiols, such as primary thiols, are particularly preferred, and amines, such as secondary amines, can also be preferred, as these form nitrosothiols and diazeniumdiolates. These functional groups can each be preferred for various embodiments, with nitrosothiols preferred when sustained nitric oxide release over extended periods of time, such as multiple days to multiple weeks, is desired, and diazeniumdiolates preferred when nitric oxide release over a relatively shorter period of time, i.e., minutes or hours, is desired.

To prepare a nitrosylated polymer, a polymer with a multiplicity of pendant nucleophilic groups is reacted with a nitrosylating agent under conditions suitable for nitrosylating the nucleophilic groups. To prepare a nitrated polymer, a polymer with a multiplicity of pendant nucleophilic groups is reacted with a nitrating agent under conditions suitable for nitrating the nucleophilic groups.

The preparation of nitrated and nitrosylated polymers will now be described with respect to S-nitrosylated and S-nitrated polymers. It should be understood that the procedures described herein for the preparation of S-nitrosylated and S-nitrated polymers can be used for the nitration or nitrosylation of polymers with pendant nucleophilic groups other than thiols, as described above. Although some variation in conditions may be required, such modification can be determined by one of ordinary skill in the art with no more than routine experimentation.

S-nitrosylated polymers and S-nitrated polymers can be prepared from polymers having a multiplicity of pendant thiol groups, referred to herein as “polythiolated polymers”. To prepare an S-nitrosylated polymer, a polythiolated polymer is reacted with a nitrosylating agent under conditions suitable for nitrosylating free thiol groups. To prepare an S-nitrated polymer, a polythiolated polymer is reacted with a nitrating agent under conditions suitable for nitrating free thiol groups.

Suitable nitrosylating agents and nitrating agents are disclosed, for example, in Feelisch and Stamler, “Donors of Nitrogen Oxides”, Methods in Nitric Oxide Research edited by Feelisch and Stamler, (John Wiley & Sons) (1996), the teachings of which are hereby incorporated into this application. Suitable nitrosylating agents include acidic nitrite, nitrosyl chloride, compounds comprising an S-nitroso group (S-nitroso-N-acetyl-D,L-penicillamine (SNAP), S-nitrosoglutathione (SNOG), N-acetyl-S-nitrosopenicillaminyl-S-nitrosopenicillamine, S-nitrosocysteine, S-nitrosothioglycerol, S-nitrosodithiothreitol and S-nitrosomercaptoethanol), an organic nitrite (e.g. ethyl nitrite, isobutyl nitrite, and amyl nitrite) peroxynitrites, nitrosonium salts (e.g. nitrosyl hydrogen sulfate), oxadiazoles (e.g. 4-phenyl-3-furoxancarbonitrile) and the like. Suitable nitrating agents include organic nitrates (e.g. nitroglycerin, isosorbide dinitrate, isosorbide 5-mononitrate, isobutyl nitrate and isopentyl nitrate), nitronium salts (e.g. nitronium tetrafluoroborate), and the like.

Nitrosylation with acidic nitrite can be, for example, carried out in an aqueous solution with a nitrite salt, e.g. NaNO₂, KNO₂, LiNO₂ and the like, in the presence of an acid, e.g. HCl, acetic acid, H₃PO₄ and the like, at a temperature from about −20° C. to about 50° C., preferably at zero degrees Celsius. Generally, from about 0.8 to about 2.0, preferably about 0.9 to about 1.1 equivalents of nitrosylating agent are used per thiol being nitrosylated. Sufficient acid is added to convert all of the nitrite salt to nitrous acid.

Polythiolated polymers can be formed from polymers having a multiplicity of pendant nucleophilic groups, such as alcohols or amines. The pendant nucleophilic groups can be converted to pendant thiol groups by methods known in the art and disclosed in Gaddell and Defaye, Angew. Chem. Int. Ed. Engl. 30: 78 (1991) and Rojas et al., J. Am. Chem. Soc. 117: 336 (1995), the teachings of which are hereby incorporated into this application by reference.

In one embodiment, the S-nitrosylated polymer is an S-nitrosylated polysaccharide. Polythiolated and perthiolated polysaccharides can be nitrosylated in the presence of a suitable nitrosylating agents such as acidic nitrite or nitrosyl chloride, as described elsewhere herein.

It is to be understood that agents capable of nitrosylating a free thiol, in some instances, also oxidize free thiols to form disulfide bonds. Thus, treating a polythiolated polymer (e.g. polythiolated polysaccharides such as polythiolated cyclodextrins) with a nitrosylating agent, e.g. acidified nitrite, nitrosyl chloride, S-nitrosothiols can, in some instances, result in the formation of a crosslinked S-nitrosylated polymer matrix. A “polymer matrix” is a molecule comprising a multiplicity of individual polymers connected or “crosslinked” by intermolecular bonds. Thus, in some instances the nitrosylating agent nitrosylates some of the thiols and, in addition, crosslinks the individual polymers by causing the formation of intermolecular disulfide bonds. Such polymer matrices are encompassed by the term “S-nitrosylated polymer” and are included within the scope of the present invention. When an excess of the nitrosylating agent is used and when the nitrosylating agent is of a sufficient size, it can be incorporated, or “entwined,” within the polymeric matrix by the intermolecular disulfide bonds which crosslink the individual polymer molecules, thereby forming a complex between the polymer and the nitrosylating agent.

S-nitrosylated polysaccharides, in particular S-nitrosylated cyclized polysaccharides such as S-nitrosylated cyclodextrins, can form a complex with a suitable nitrosylating agent when more than one equivalent of nitrosylating agent with respect to free thiols in the polythiolated polysaccharide is used during the nitrosylation reaction, as described above. Generally, between about 1.1 to about 5.0 equivalents of nitrosylating agent are used to form a complex, preferably between about 1.1 to about 2.0 equivalents.

### Particles

In some embodiments, where particles comprising NO-releasing functional groups are embedded inside of polymers used to form polymeric coatings, or the medical device is itself a particle comprising NO-releasing functional groups, the particles can be microparticles or nanoparticles. These particles can be formed from polymers that comprise NO-releasing functional groups, or can include small molecules that comprise NO-releasing functional groups.

As discussed below, in some embodiments, the conditions used to form particles often involve subjecting polymers to conditions which decompose NO-releasing functional groups, at least at some rate, for example, by exposing the particles and their components to heat, light, and/or pH levels below around 7. Accordingly, in some embodiments, the particles are prepared using polymers and/or compounds with pendant functional groups that can be converted to NO-releasing compounds, and these groups are converted to NO-releasing functional groups after the particles are formed. In other embodiments, the particles are prepared using polymers comprising NO-releasing functional groups, and it is understood that the particles may lose some of their NO-releasing capacity due to decomposition of a fraction of the NO-releasing functional groups during particle formation.

Microparticles are typically defined as particles between 0.1 and 100 m in diameter, and nanoparticles are typically defined as particles between 1 and 100 nm in diameter. When used in drug delivery, the particles are preferably prepared from biodegradable polymers, such as polylactic acid, polyglycolic acid, PLGA, polycaprolactone, polyanhydrides, and other polymers described elsewhere herein.

Microparticles or nanoparticles can be prepared via a wide variety of methods. Representative techniques for preparing microparticles include emulsion-solvent evaporation (oil/water, water/oil, and water/oil/water), phase separation (non-solvent addition and solvent partitioning), interfacial polymerization, spray drying, emulsion extraction processes, milling techniques, such as jet milling techniques, fluidization, and solvent precipitation methods. The processes often involve drying the particles to remove the solvents used in their preparation, and the drying processes typically involve using heat that would be sufficient to decompose the NO-releasing functional groups, and cause them to release nitric oxide prematurely (i.e., before they are implanted into a patient).

Accordingly, in one embodiment, functional groups like thiols and amines are converted into NO-releasing functional groups after the particles are formed, so this premature decomposition can largely be avoided. Given their relatively large surface area, it is relatively easy to convert the functional groups into NO-releasing functional groups.

The particles ideally comprise biodegradable polymers. Many biodegradable polymer systems are generally accepted as safe for human use, which results in a broad set of NO donor and biodegradable polymer combinations.

Creating a controlled-release biodegradable particle avoids the undesirable condition of leaving potentially harmful materials behind after an implanted device, particularly percutaneous implants such as a port, catheter, and the like, have been removed from the host.

Further, this approach facilitates a broad range of hydrophobic particles within which one can place the NO-releasing donor compound. An example of a desirable embodiment can be the doping of thiolactic acid (the donor) within biodegradable polycaprolactone or PLGA particles.

The processes for preparing microparticles often involve forming a polymer solution, and active pharmaceutical agents can be present in the solution. When the particles precipitate out of solution, the active pharmaceutical agents can be incorporated into the polymers.

In those embodiments where the pH of the solution is not overly acidic (i.e., is around 7.0 or higher), and the temperature at which the particles are formed, and, subsequently, isolated, is not sufficient to significantly decompose the NO-releasing functional groups, the NO-releasing functional groups on the polymers are not significantly decomposed (i.e., less than 10% loss of NO-releasing capacity during particle formation).

In those embodiments where the pH of the solution is relatively acidic (i.e., below around 6.9), and/or the temperature at which the particles are formed, and, subsequently, isolated, is sufficient to significantly decompose the NO-releasing functional groups, the NO-releasing functional groups on the polymers are not significantly decomposed (i.e., less than 10% loss of NO-releasing capacity during particle formation). The temperature at which these NO-releasing functional groups decompose varies depending on the particular functional group, but those of skill in the art can readily determine which functional groups can survive the particle formation without significant decomposition of the NO-functional groups, without undue experimentation.

Nanoparticles can be prepared by “wet” chemical processes, in which solutions of suitable compounds are mixed or otherwise treated to form an insoluble precipitate of the desired material. The size of the particles of the latter is adjusted by choosing the concentration of the reagents and the temperature of the solutions, and by adding suitable inert agents that affect the viscosity and diffusion rate of the liquid. With different parameters, the same general process may yield other nanoscale structures of the same material, such as aerogels and other porous networks.

As with other particle preparation methods, whether or not the nanoparticles are prepared using polymers and/or small molecules with NO-releasing functional groups, or with functional groups that are later converted to NO-releasing functional groups, depends largely on the temperature and pH of the solution in which the nanoparticles are prepared.

Nanoparticles formed by this method can be separated from the solvent and soluble byproducts of the reaction, typically by evaporation, sedimentation, centrifugation, washing, and/or filtration. Alternatively, if the particles are meant to be deposited on the surface of a medical device, the starting solutions can be by coated on that surface, for example, by dipping or spin-coating, and the reaction can be carried out in place.

The wet chemical approach allows fine control of the particle's chemical composition, and various additives, for example, active pharmaceutical agents, can be introduced in the reagent solutions and end up uniformly dispersed in the final nanoparticulate product.

Nanoparticles can also be prepared from macro- or micro-scale particles by grinding them in a ball mill, a planetary ball mill, or other size-reducing mechanism until enough of them are in the nanoscale size range. The resulting powder can be air classified to extract the nanoparticles.

In some embodiments, an active pharmaceutical agent can be present in the coatings, whether by blending it into the polymer coating solution, or incorporating it into micro- or nanoparticles.

### Small Molecules

Ideally, small molecules have a molecular weight of less than 1,000, more preferably less than 750, and still more preferably, less than 500 g/mol. Representative small molecules include compounds with an S-nitroso group. Examples include S-nitrosothiolacetic acid, S-nitroso-N-acetyl-D,L-penicillamine (SNAP), S-nitrosoglutathione (SNOG), N-acetyl-S-nitrosopenicillaminyl-S-nitrosopenicillamine, S-nitrosocysteine, S-nitrosothioglycerol, S-nitrosodithiothreitol, and S-nitrosomercaptoethanol.

Additional small molecules include organic nitrites (e.g. ethyl nitrite, isobutyl nitrite, and amyl nitrite), oxadiazoles (e.g. 4-phenyl-3-furoxancarbonitrile), peroxynitrites, nitrosonium salts and nitroprusside and other metal nitrosyl complexes (See Feelisch and Stamler, “Donors of Nitrogen Oxides,” Methods in Nitric Oxide Research edited by Feelisch and Stamler, (John Wiley & Sons) (1996).

The NO delivery times and delivery capacity of the S-nitrosylated polymers described herein can be increased by incorporating small molecules that include S-nitrosyl groups. The extent and degree to which delivery times and capacity are increased is dependent on the capacity of the small molecules.

Formation of Nitrosothiols and/or Diazeniumdiolates

Nitrosation conditions are disclosed, for example, in C Zhang et al. Chem. Commun., 2017, 53, 11266-11277.

Nitrosylating agents which can complex with an S-nitrosylated cyclic polysaccharide include those with the size and hydrophobicity necessary to form an inclusion complex with the cyclic polysaccharide. An “inclusion complex” is a complex between a cyclic polysaccharide such as a cyclodextrin and a small molecule such that the small molecule is situated within the cavity of the cyclic polysaccharide. The sizes of the cavities of cyclic polysaccharides such as cyclodextrins, and methods of choosing appropriate molecules for the preparation of inclusion complexes are well known in the art and can be found, for example, in Szejtli Cyclodextrins In Pharmaceutical, Kluwer Academic Publishers, pages 186-307, (1988) the teachings of which are incorporated herein by reference.

Nitrosylating agents which can complex with an S-nitrosylated cyclic polysaccharide also include nitrosylating agents with a sufficient size such that the nitrosylating agent can become incorporated into the structure of the polymer matrix of an S-nitrosylated polysaccharide. As discussed earlier, in certain instances nitrosylation of polythiolated polymers can also result in the crosslinking of individual polymer molecules by the formation of intermolecular disulfide bonds to give a polymer matrix. Suitable nitrosylating agents are those of an appropriated size such that the nitrosylating agent can be incorporated into this matrix. It is to be understood that the size requirements are determined by the structure of each individual polythiolated polymer, and that suitable nitrosylating agents can be routinely determined by the skilled artisan according to the particular S-nitrosylated polymer being prepared.

Representative nitrating agents include organic nitrates and nitronium salts.

Polymers, particles and small molecules comprising pendant nitrosothiol groups can be prepared by reacting a solution comprising a polymer, a particle, or a small molecule comprising pendant (free) thiol groups with a nitrosylating agent under conditions suitable for nitrosylating the free thiol groups.

In one embodiment, the nitrosylating agent is selected from the group consisting of an S-nitrosothiol, an organic nitrite, an oxadiazole, a peroxynitrite, a nitrosonium salt and a metal nitrosyl complex.

In one embodiment, the nitrosylating agent is an acidified nitrite. Examples of nitrites include sodium nitrite, potassium nitrite, calcium nitrite, and ammonium nitrite any soluble nitrite salt that provides a nitrite anion to the nitrosylation solution. Examples of acids include hydrochloric acid, hydrobromic acid, hydroidic acid, sulfuric acid, phosphoric acid, and any strong acid that facilitates the conversion of the nitrite anion into a gaseous nitrosylating agent.

In some embodiments, the polymer, particle or small molecule is reacted with between about 0.8 and about 2.0 molar equivalents, ideally between about 0.9 and about 1.1 molar equivalents, of acidified nitrite per mole of free primary thiol or secondary amine.

In other embodiments, the nitrosylating agent is nitrosyl chloride.

In some embodiments, the nitroxylating agent is gaseous. In some aspects of these embodiments, An “effective amount” of a gaseous, nitrosylating agent is the quantity that results in nitrosylation of at least around 50%, preferably at least around 75%, and more preferably, at least around 90% of the free primary thiol or secondary amine groups in the compound, particle or polymer.

Preferably, a sufficient amount of the gaseous, nitrosylating agent is used to saturate the free primary thiol or secondary amine groups in the compound, particle or polymer with NO, i.e. all or substantially all (i.e., greater than 90%) of the primary thiol or secondary amine groups become nitrosylated to form nitrosothiols or diazeniumdiolates, respectively. An effective amount ranges from about 0.8 atmospheres to about 10 atmospheres, and is preferably about one atmosphere.

### Representative Nitrosylation Conditions

In one embodiment, polymers comprising primary thiol or secondary amine groups are dissolved in aqueous hydroxide solutions, such as NaOH solutions, and a mixture of an aqueous nitrite, such as NaNO₂, ideally around 0.5 to 1.5 equivalents per mole of free thiol or amine) and an acid, such as HCl, is added. Where this process produces a precipitate, the precipitate can be collected, and the acidic supernatant removed. The precipitate can be washed with water, such as deionized water, ideally with agitation or stirring, until the supernatant has a pH of around 6-8.

In another embodiment, a polymer comprising primary thiol and/or secondary amine groups can be dissolved in an appropriate solvent, for example, a polar aprotic solvent such as DMF or DMSO. Nitrosyl chloride can be bubbled through the solution, and the solvent removed in vacuo or under a stream of an inert gas, such as nitrogen or argon, to afford a polymeric product comprising NO-releasing groups.

In still another embodiment, a polymer comprising primary thiol or secondary amine groups is dissolved in a hydroxide solution, such as an NaOH solution. A nitroso compound, such as D(+)-S-nitroso-N-acetylpenicillamine, can be added, and this typically forms a precipitate. The precipitate can be collected and washed, typically until the supernatant has a pH between around 6 and 8.

While not wishing to be bound by any particular theory, it is believed that some of the nitrating agent or nitrosylating agent will contact the polymer, and, where the agent is gaseous, will contact the polymer at more than just the surface, and nitrate or nitrosylate the functional groups capable of being converted to NO-releasing groups. This generates the NO₂ or NO capacity of the polymer, after fabrication conditions are used which might otherwise result in a loss of NO-releasing capacity if the NO-releasing groups were formed before the medical device was fabricated.

### Formation of Polymeric Coatings

Polymeric coatings can be hydrophobic or hydrophilic. In some embodiments, particularly where NO release is desired over an extended period of time, it can be desirable to use hydrophobic coatings, particularly when these coatings comprise embedded particles or small molecules that include NO-releasing functional groups.

By using hydrophobic coatings, physiological fluids cannot rapidly penetrate the coating and access the embedded particles or small molecules, and this can extend the time during which NO can be released. In other embodiments, the coatings are hydrophilic, which can help improve biocompatibility and lessen the foreign body response, and, when exposed to physiological fluids, can enhance the rate at which NO can be released, relative to hydrophobic polymers. In various aspects of each of these embodiments, the polymers are biodegradable.

The particles embedded in or mixed in the polymers can be biodegradable, and can be formed of the same or different polymers used to form the coating. Where the particles are biodegradable, the rate of NO release can be controlled by controlling the degradation rate of the polymer. As more of the particle surface is exposed, more of the NO-releasing functional groups are exposed to physiological fluids, and thus release more NO.

In some embodiments, a hydrophobic polymer is used for the coating, and hydrophilic or amphiphilic particles are embedded in the polymer coating. In other embodiments, a hydrophobic polymer is used for the coating, and hydrophobic particles are embedded in the polymer coating. In still other embodiments, a hydrophilic or amphiphilic polymer is used for the coating, and hydrophobic particles are embedded in the polymer coating. In still other embodiments, a hydrophilic or amphiphilic polymer is used for the coating, and hydrophilic or amphiphilic particles are embedded in the polymer coating.

The loading of the particles in the polymeric coating can vary, and is typically in the range of between about 1 and about 50% by weight, more typically, between about 5 and about 45% by weight, and preferably between about 10 and about 40% by weight.

There are a number of ways the coatings can be applied. Each method has inherent benefits and detriments, and not all methods are applicable to all devices or materials. However, those of skill in the art will readily appreciate which coating methods are best for which devices.

Dip coating is a common process for coating medical devices. Dip coating typically involves surface preparation/washing, submersing the device in a coating liquid, removing the device from the liquid, drying and/or curing the coating, using heat or light, such as UV light, and post-processing.

As discussed above with respect to particle formation, if the submersion/immersion, drying and/or curing steps might result in significant decomposition of the NO-releasing functional groups if such were present on the polymer, and/or in particles or small molecules embedded in the polymer at the time the coating is applied, then in some embodiments, functional groups like primary thiols and secondary amines are present in the polymers, particles, or small molecules, and are then converted to NO-releasing functional groups after the coating is applied, thus avoiding decomposition of the NO-releasing functional groups while the coating is applied and/or cured.

Spray coating can also be used. Spray coating typically involves using a nozzle and driver to nebulize a coating solution and apply it to the surface of the medical device as a mist. Ultrasound transducers can be used control spray droplet size, which impacts the thickness and quality of the coating.

Reel-to-reel coatings can be used in certain embodiments, though not typically with small, intricate devices. A reel of wire or film is unraveled and travels through a reservoir of a coating solution, and then into an oven for drying or curing before being rolled up onto a second reel. This approach can be used for preparing the tapes and monoliths described herein.

As with dip coating, in those embodiments in which the drying and/or curing steps would likely significantly decompose NO-releasing functional groups present on the polymer used for the coating, or in particles or small molecules mixed in or embedded in the polymers, it may be desirable to convert functional groups like primary thiols or secondary amines to NO-releasing groups after the coating is applied.

Robotic coating is applicable for complicated shapes and is amenable to a continuous system. Tiny nozzles are directed robotically to trace along struts and other structures. The viscosity of the coating solution can be programmed as needed.

Spin coating is another common technique for applying thin films to substrates. Although it quickly and easily produces uniform films, ranging from a few nanometers to a few microns in thickness, it is typically only used to coat flat surfaces.

Submersion coating is a relatively simple process. Many devices require only a 30-second submersion to be fully functional, and often require no cure. Submersion coating is commonly used to coat medical devices.

In one embodiment, a medical device, for example an electrode used in a continuous glucose monitor, or a stent, is coated with a polymer that comprises NO-releasing functional groups, or, in certain aspects of this embodiment, functional groups, such as primary thiols or secondary amines, that can be converted to NO-releasing functional groups after the coating process is complete.

In another embodiment, the medical device is coated with a polymer that includes embedded particles or small molecules, where the particles or small molecules comprise NO-releasing functional groups, or, in certain aspects of this embodiment, functional groups, such as primary thiols or secondary amines, that can be converted to NO-releasing functional groups after the coating process is complete. In other embodiments, a polymer and embedded particles and/or small molecules include NO-releasing groups, or, in certain aspects of these embodiments, functional groups that can be converted to NO-releasing groups after the coating process is complete.

In one aspect of these embodiments, the device is coated with a polysaccharide comprising pendant nitrosothiol groups, or pendant thiol groups which are then converted to NO-releasing groups, thus forming S-nitrosylated cyclodextrins, starches, dextrins, dextrans, glycosaminoglycans, celluloses, and the like.

In one embodiment, a medical device is coated with a polymer solution comprising a polysaccharide comprising multiple nitrosothiol groups (i.e., polythiolated polysaccharide that, before or after the coating step is completed, is contacted with a nitrosylating agent (or nitrosating agent) under conditions suitable for nitrosylating (or nitrating) free thiol groups, resulting in formation of an S-nitrosylated (or S-nitrosated) polysaccharide.

In one aspect of this embodiment, a polymeric coating solution comprises a polar, aprotic solvent such as dimethylformamide (DMF) or dimethylsulfoxide (DMSO). The coating solution is applied to all or part of the medical device, using any of the conventional methods for applying a coating solution that is appropriate for the device. The coating can then be dried in vacuo, in an oven, or under a stream of an inert gas such as nitrogen or argon.

In those embodiments where the coating comprises functional groups that can later be converted to NO-releasing functional groups, the coated device can then be subjected to appropriate conditions to convert pendant thiol or amine groups in the coating to NO-releasing groups.

In one aspect of these embodiments, the polymer solution comprises a hydrophobic polymer, such as polyurethane, and particles of a hydrophilic or hydrophobic polymer, where the particles comprise NO-releasing functional groups, such as nitrosothiols or diazeniumdiolates, or comprise functional groups, such as primary thiols or secondary amines, that are later converted to NO-releasing functional groups, such as nitrosothiols or diazeniumdiolates. The coating solution is applied to the medical device, or a portion thereof, and the coating is cured. In those embodiments where the coating comprises functional groups that can later be converted to NO-releasing functional groups, the coated device can then be subjected to appropriate conditions to convert pendant thiol or amine groups in the coating to NO-releasing groups.

In any of these embodiments, the coating can optionally include a dye, pigment, and/or light-stabilizing compound, which inhibits decomposition of the NO-releasing groups when the coated medical devices are exposed to light. In another aspect, the sprays, tapes, monoliths, and the like can also include such dyes, pigments and/or light-stabilizing compounds.

### Monoliths/Films/Tapes

In various embodiments, a tape or film is formed from a biodegradable polymer comprises one or more pendant NO-releasing groups, and/or comprises embedded particles or small molecules which comprise NO-releasing groups. In some aspects of this embodiment, the tapes/films coat all or a portion of medical devices.

The tape or film can be physically or chemically attached to a medical device, such as an implant. Where the tape or film is chemically attached, it preferably comprises a biocompatible, and preferably biodegradable, adhesive. Such adhesives are well-known to those of skill in the art. One and two-part epoxy and silicone biocompatible adhesives can be used, as can various light-cured materials, epoxy-polyurethane blends, and cyanoacrylates. In one embodiment, the adhesive is a biocompatible and biodegradable polyurethane adhesive. In another embodiment, the adhesive is a poly (glycerol sebacate acrylate) (PGSA).

### Sprayable Formulations

The sprayable formulations described herein comprise mixtures of chemicals that form a biodegradable polymeric film on at least one surface, or portion thereof, of an implant to which they are applied. In one embodiment, the polymeric film conformably adheres to the covered area on the implant.

The formulations include a biodegradable, biocompatible polymer, a solvent for the polymer, which solvent has a boiling point below around 100° C., preferably below around 85° C., and more preferably below around 70° C., and a propellant, which can be a gas or a volatile liquid. The polymer can be any of the polymers described elsewhere herein, including those with pendant NO-releasing functional groups, and, in one embodiment, is a hydrophobic polymer. Ideally, the polymers have relatively low cytotoxicity. In some embodiments, the polymer does not include pendant NO-releasing groups, and in those embodiments, the sprayable formulations include particles with NO-releasing groups, or small molecules with pendant NO-releasing groups.

The spray formulation has one or more of the following properties:
(1) low viscosity or liquid-like properties when sprayed, to enable easy application to a desired area on the implant to which it is applied,
(2) minimum washout by body fluids and activation of the NO-releasable functional groups only when the medical device is implanted,
(3) significant adhesive strength, especially in the presence of blood and/or other body fluids,
(4) ability to resist the mechanical loads to which the implant is subjected following implantation,
(5) minimal inflammatory response, and
(6) biodegradability.

In addition to these properties, the film formed by the sprayable formulation releases nitric oxide over time. The release of nitric oxide minimizes microbial contamination that often accompanies surgery, promotes wound healing, increases vascularization around the implant, and minimize scar formation. The sprayable formulations described herein provide this nitric oxide release.

In one embodiment, the biodegradable polymer in the sprayable formulation comprises one or more of (meth)acrylate functional groups, or cyanoacrylates, or a combination of albumin and glutaraldehyde, or includes poly(ethylene glycol) (PEG) blocks, or includes polyurethane or fibrin.

Cyanoacrylates belong to a class of monomers consisting of the alkyl esters of 2-cyanoacrylic acid. Representative cyanoacrylates include methyl, ethyl, n-butyl, isobutyl, isohexyl and octyl cyanoacrylates. Cyanoacrylates are capable of adhering to most implant surfaces.

Fibrin can be obtained, for example, from pooled human plasma.

Where cyanoacrylate (CA) or fibrin are used, they may not have all or most of the desired properties for a sprayable formulation. However, particles or small molecules that release nitric oxide can be blended with these materials, and at least they can have the beneficial properties associated with nitric oxide release.

In one embodiment, the sprayable formulation comprises a combination of purified bovine serum albumin (BSA) and glutaraldehyde, which polymerizes in situ at the application site within 30 seconds, with full strength achieved in 2 minutes.

In another embodiment, the sprayable formulation comprises a hyperbranched polyurethane with isocyanate end groups, and lysine. The amine groups in the lysine crosslink with the isocyanate groups, with adhesive crosslinking taking place within 25 minutes.

PEG-based polymers can also be used. These polymers can be polyethylene glycol-based synthetic hydrogels, which comprise a block copolymer including one or more polyethylene glycol blocks and one or more PLGA blocks, which also include carbonate linkages, and which include (meth)acrylate end caps. The presence of a PEG block in the polymer can minimize tissue adhesion to the implanted medical device. The film is degradable by virtue of the PLGA block. It can also be adhered to implant using the (meth)acrylate terminal end groups. In one embodiment, the sprayable formulation comprises a PEG-co-trimethylene carbonate-co-lactide with acrylated end groups, and eosin Y is added as a component to react with light after the formulation is applied to a medical device, to produce the free radicals that polymerize the polymer in situ.

In another embodiment, a polymer which comprises human serum albumin (HSA) and di-PEG-succinimidyl succinate, which crosslink with each other, and which can be sprayed administered using a dual nozzle sprayer to avoid having the components mix before application, so they can be cured in situ by crosslinking upon application.

In another embodiment, the sprayable formulation comprises tetra-PEG-succinimidyl ester and trilysine amine, which can be administered using a dual nozzle sprayer, and crosslink when applied.

In one embodiment, the sprayable formulation comprises a block copolymer comprising one or more polyalkylene glycol blocks, such as polyethylene glycol blocks, and one or more degradable blocks.

In some embodiments, the degradable blocks are formed from any suitable combination of degradable monomeric units, such as lactic acid, glycolic acid, hydroxybutyric acid, caprolactone, carbonates, and the like, and in some embodiments, are a peptide such as trilysine or other short chain (i.e., less than 25 monomeric units) peptide that comprises more than two lysine monomeric units, or a protein, such as albumin.

In some embodiments, the polymer in the sprayable formulation also comprises a vinyl group (such as a (meth)acrylate group) that can be polymerized via free radical polymerization. In other embodiments, the sprayable formulation is a two or more component system, where one component includes a functional group that can crosslink with a functional group on another component.

In one embodiment, a polyalkylene glycol, such as polyethylene glycol, block comprises a functional group that crosslinks with a different functional group on a degradable block. Those of skill in the art understand what functional groups are capable of crosslinking with other functional groups under physiological conditions.

In one aspect of this embodiment, the degradable blocks comprise one or more monomeric units that comprise pendant thiol or amine groups, which can be modified to form nitrosothiol, diazeniumdiolate, or other NO-releasing groups before the glue is applied. In this embodiment, it is important that the nitrosothiol, diazeniumdiolate, or other NO-releasing functional groups do not interfere with the crosslinking chemistry. In another embodiment, the surgical glue is blended with particles or small molecules that comprise NO-releasing functional groups. Combinations of these approaches can be used.

The polymers can be modified to include monomer units with pendant thiol or amine groups, which can be converted to nitrosothiol groups, diazeniumdiolate groups, or other NO-releasing groups, and thus provide NO release after the sprayable formulation is applied, and the resulting film coating is exposed to physiological fluids. Alternatively, the polymers in the sprayable formulation can be blended with particles or small molecules that comprise NO-releasing functional groups.

### Optional Additional Active Agents

The coatings, or the tapes, monoliths, and/or films formed from applying the sprayable formulations to a medical device, can also release additional substances over time that inhibit the foreign body response by ways other than local NO release. These substances include VEGF or VEGF promoters, TNF-α and/or β inhibitors, including anti-TNF-α and/or β antibodies, halofunginone, antimicrobial compounds, anti-inflammatory compounds, such as dexamethasone and monobutyrin. These additional substances can further prevent or minimize the foreign body response.

### Miscellaneous

In one embodiment, particles comprising biodegradable polymers encapsulate a NO donor moiety, such as a polymer or small molecule comprising pendant NO-releasing functional groups. This is advantageous for multiple reasons. Many biodegradable polymer systems that are generally accepted as safe for human use. This results in a broad set of combinations of NO donors and biodegradable polymers.

Creating a controlled-release biodegradable particle avoids the undesirable condition of leaving potentially harmful materials behind after a device, such as a glucose sensor, has been removed from the host.

Further, this approach facilitates a broad range of hydrophobic particles within which one can place the NO-releasing compound. One example of an NO-releasing compound is thiolactic acid, where the thiol group has been converted to a nitrosothiol group, where this modified thiolactic acid is embedded within a biodegradable particle, such as biodegradable polycaprolactone or PLGA particles.

Applications include the outer hydrophilic coatings of an analyte sensor, such as a glucose sensor, or the formulation of a surgical glue. In the first example, the reduced FBR on the glucose sensor will extend its clinically useful lifetime. In the second example, the NO can improve healing rates and decrease scar tissue formation, which can be desirable, since scar tissue created after a successful surgical outcome can often lead to long-term secondary complications.

### Medical Devices

In some embodiments, the medical devices are percutaneous implants that comprise a coating that includes biocompatible, and, in some cases, biodegradable polymers, and optionally includes embedded particles. Representative percutaneous implants include percutaneous glucose monitors, catheters, including urinary catheters and venous ports/catheters for chemotherapy (e.g port-a-cath), fluid-draining devices (drains), drug delivery devices, blood-sampling devices, and percutaneously implanted neurostimulator electrode arrays, tracheal stomal ports, abdominal stomal ports, and any device that punctures the skin for the purpose of accessing bodily fluids below the skin's surface or bodily cavities.

In other embodiments, a medical device is a tape, stitch, glue, monolith, or tissue scaffold, and other devices that are applied to, rather than being implanted in, a patient, or which can be used to grow cells, tissues, or organs ex vivo.

In some embodiments of these devices, the devices comprise an NO-releasing coating that includes a polymer with pendant NO-releasing groups, in other embodiments, the devices comprise a coating that incorporates particles or small molecules that include NO-releasing groups, and in still other embodiments, the coating comprise a polymer with pendant NO-releasing groups, and also comprises embedded particles or small molecules which include NO-releasing groups.

In various aspects of these embodiments, an NO-releasing coating can be formed of a biocompatible polymer, which can optionally be a biodegradable polymer, comprising pendant NO-releasing groups, such as diazenium diolates and/or nitrosothiol (SNO) groups, and a dye, pigment, or light stabilizing compound. Before implantation, the dye, pigment or light stabilizing compound minimizes degradation of the NO-releasing groups due to light exposure. Following implantation, the NO-releasing groups on the polymer surface are exposed to biological fluids, which cause the groups to release nitric oxide. The biocompatible, optionally biodegradable, polymers can be hydrophilic or hydrophobic. Where the polymer is a biodegradable polymer, in order to delay degradation, and extend the release of nitric oxide, it can be preferred that the polymer be hydrophobic.

In other aspects of this embodiment, the coating may or may not include NO-releasing groups, such as diazenium diolate and/or nitrosothiol groups, but comprises embedded particles, such as micro or nano-particles, which are prepared from small molecules and/or polymeric compounds which comprise nitrosothiol groups.

In still other aspects of this embodiment, the coating may include small molecules and/or polymeric compounds that comprise nitrosothiol groups, which are not in the form of microparticles or nanoparticles, but rather, are simply blended into the polymer.

The coating can be applied, for example, by dip coating, spraying, brushing, and the like. This second coating minimizes degradation of the NO-releasing groups. Ideally, the coating is permeable to physiological fluids when the device is implanted, such that nitric oxide can be released from the coating.

In various aspects of this embodiment, the coating can be formed of a biocompatible, optionally biodegradable polymer that comprises pendant NO-releasing groups, such as diazenium diolate and/or nitrosothiol (SNO) groups.

Following implantation, NO-releasing groups, such as nitrosothiol groups on the polymer surface, are exposed to hydrophilic biological fluids, which cause the groups to release nitric oxide. As the polymer surface biodegrades, a fresh polymer surface is continuously exposed. Depending on the hydrophobicity of the polymer, once the polymer is exposed to moisture, both interior and surface NO-releasing groups may deliver their NO payload, or the interior NO-releasing groups may be protected, and the polymer may continuously release nitric oxide as the polymer biodegrades. Accordingly, nitric oxide may be released until the coating completely biodegrades. When the polymer is a hydrophobic polymer, it can provide extended nitric oxide release relative to hydrophilic polymers, whether or not pendant NO-releasing groups are present on the polymer, or on particles embedded within the polymer.

In some embodiments, the devices comprise a coating formed of a biocompatible polymer, which is optionally a biodegradable polymer, that comprises pendant NO-releasing groups, such as diazeniumdiolate and/or nitrosothiol (SNO) groups. Following implantation, NO-releasing groups on the polymer surface are exposed to biological fluids, which cause the groups to release nitric oxide.

In various embodiments, the polymers and/or compounds include nitrosothiol groups are capable of releasing nitric oxide over an extended period of time, for example, over a week, two weeks, three weeks, or even a month or more.

The degradation time can be controlled by judicious selection of the monomers used to prepare the biodegradable polymers, as well as the percent crystallinity, molecular weight, and hydrophobicity. For example, polyglycolic acid tends to biodegrade faster than polylactic acid, and copolymers of lactic and glycolic acid can be prepared with varying ratios of these monomers, where the degradation time can be controlled.

In still other embodiments, the devices are sprayed with an NO-releasing formulation as described herein, or an NO-releasing tape or monolith is applied to the devices.

### Percutaneous Implants

In some embodiments, the medical devices are percutaneous implants, and all or a portion of the implant is formed of a material that releases nitric oxide, is sprayed or coated with a material that releases nitric oxide, or a tape or monolith is applied to the implant, where the tape or monolith releases nitric oxide. Percutaneous implants include, for example, percutaneous glucose monitors, catheters/ports, including urinary catheters and venous ports/catheters for chemotherapy (e.g., port-a-cath), as well as stomal ports, such as tracheal and abdominal stomal ports, fluid-draining devices (i.e., drains), drug delivery devices, percutaneously implanted neurostimulator electrode arrays, blood sampling devices, and any other device which punctures the skin for the purpose of accessing bodily fluids below the skin's surface or within body cavities.

Catheters, ports, fluid-draining devices (i.e., drains), drug delivery devices and blood sampling devices can be modified by coating with a polymeric coating as described herein, adhering a tape or monolith, or sprayed with a sprayable formulation as described herein, where the coating, tape, monolith, or sprayed-on formulation releases nitric oxide upon implantation. The release of nitric oxide can inhibit bacterial growth in and around the devices, and inhibit the foreign body response to the devices. By way of example, urinary catheters can cause urinary tract infections, and the release of nitric oxide from the catheters can minimize the likelihood of infection.

Fluid-draining device (e.g., drains) can be used, for example, to drain ascites fluid or fluid that builds up around a patient's heart, or fluid that builds up around a surgical site, and the release of nitric oxide can minimize microbial contamination and promote wound healing.

Drug delivery and blood sampling devices typically include a tube that is inserted into a patient for delivering a drug over an extended period of time or taking repeated blood samples. Examples include ports, such as chest ports. The tissue surrounding these ports can be subject to infection and/or the foreign body response, which can be minimized using the coatings, tapes, monoliths, or sprayable formulations described herein.

### Percutaneous Glucose Monitors

A conventional glucose monitoring system measures insulin levels when a drop of blood is collected on a strip, which strip is inserted into the system. This type of device is used, periodically, to confirm that the “flash” glucose monitoring system is calibrated correctly. This can be important over time, as the body develops a foreign body response to the injected electrode.

A “flash” glucose monitoring system typically includes a wireless transmitter for wirelessly transmitting data on a patient's insulin levels to a display. Conventional glucose monitoring systems that do not provide ways to counter the foreign body response, as well as the glucose monitoring systems described herein, which do provide ways to counter the foreign body response.

The system also typically includes a surface that adheres to the user's skin, and includes an injectable biosensor. Because they can be miniaturized, and provide high selectivity and sensitivity governed by the specific biocatalytic reactions of an immobilized enzyme, it is preferable that the CGMs described herein use electrochemical biosensors.

The biosensor measures glucose levels, and these levels are typically displayed on a display, such as a smart phone screen. The glucose levels can be measured, for example, using a series of known chemical reactions. Glucose is oxidized, by glucose oxidase, to glucanolactone.

Oxygen is consumed, and hydrogen peroxide is produced. An Ag/AgCl electrode can determine oxygen consumption and/or hydrogen peroxide product, and these levels can be equated to the amount of glucose that was oxidized. This in turn provides a measure of glucose levels.

Enzyme-based sensors measure the rate of glucose oxidation through a change in oxygen or hydrogen peroxide concentrations upon reaction of glucose with a glucose-specific enzyme (e.g., GOx or GDH). In some embodiments, the enzyme is immobilized on the surface of the electrode. The enzyme is reduced upon converting glucose to gluconolactone. Ambient oxygen facilitates the conversion of the reduced enzyme back to its oxidized form with concomitant production of hydrogen peroxide.

The glucose concentration correlates with the amperometric signal obtained from either the electrochemical oxidation of produced hydrogen peroxide or the reduction of consumed oxygen. Although enzyme-based electrochemical glucose biosensors are characterized with high selectivity and sensitivity due their enzymatic nature, the dynamic range of such sensors is limited by co-substrate (i.e., oxygen) availability. An outer diffusion-limiting membrane is thus employed to control for this and eliminate oxygen deficiencies, albeit with a slightly delayed sensor response. Additionally, the working electrode potential required to monitor hydrogen peroxide (i.e., about +0.6 V vs. Ag/AgCl) will also oxidize electroactive endogenous species (e.g., ascorbic acid and acetaminophen) and create high current densities. To address these shortcomings, second-generation electrochemical glucose biosensors employ electron mediators (e.g., [Os(4,4′-dimethoxy-2,2′-bipyridine)2Cl]+/2+) “wired” to the enzyme on a hydrophilic polymer matrix (e.g., poly(vinylpyridine) or poly(vinylimidazole)). Such mediators are capable of shuttling electrons from the redox center of the enzyme to the surface of the electrode, thus allowing for a lower applied electrode potential. In turn, the sensor response is independent of the co-substrate and interferences.

That said, other glucose measuring techniques that use an injected biosensor can also be used, and are within the scope of the invention. For example, in one embodiment, the CGM uses a non-enzymatic electrochemical glucose sensor rather than an enzymatic electrochemical glucose sensor. In one aspect of this embodiment, glucose is measured directly via direct electro-oxidation at high-surface area (i.e., porous) platinum electrodes, or through potentiometric detection dependent on pKa changes in a conducting polymer.

An injectable (“percutaneous”) glucose sensor can include a potentiostat, an Ag/AgCl electrode, and a Pt—Ir electrode. The Pt—Ir electrode is typically coated with a number of polymer layers, including an outer-diffusion limiting layer, an enzyme (GOx) layer, and an inter-selective layer. The portion of the CGM that overlies the user's skin includes a sensor array, an electronics module, a battery, and a telemetry/transmission portal.

In one embodiment of a subcutaneous continuous glucose monitoring (CGM) device, the device includes an enzyme-immobilized amperometric biosensor. The device includes a disk-type sensor with a titanium housing. The device includes a sensor array, an electronics module, a battery, and a telemetry/transmission portal. These components are typically present in a percutaneous CGM as well, but are found in the portion of the CGM that overlies the user's skin, as only the biosensor is injected.

A display for displaying glucose levels which are wirelessly transmitted from a “flash” glucose monitoring system can receive signals from a telemetry/transmission portal.

In use, the glucose monitor is applied to the skin, and the electrode pierces the skin to a depth of between about 3 and 8 mm, more typically between about 4 and about 7 mm. The electrode comprises a coating, which releases nitric oxide at or near the surface of the electrode.

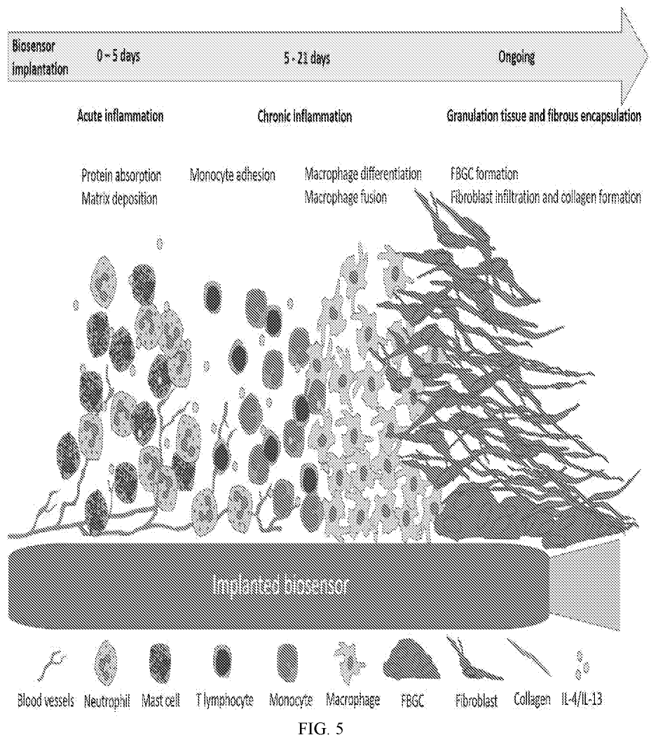

With respect to percutaneous or subcutaneous glucose monitors, the foreign body response over time is shown in FIG. 5. Over the first five days, the implanted biosensor experiences protein absorption and matrix deposition, with neutrophils, mast cells and blood vessels forming around the sensor. This is viewed as an acute inflammatory response to the implanted biosensor. Between five and twenty one days post implantation, the sensor experiences monocyte adhesion, and macrophage fusion and differentiation. This is viewed as a chronic inflammatory response. From that point on, the sensor experiences FBGC (foreign body giant cells) formation, and fibroblast infiltration and collagen formation, which forms granulation tissue and fibrous encapsulation of the implanted biosensor.

The foreign body response following implantation can also result in a local pH dropping as low as 3.6 and disrupting biosensor performance, as the activity of GOx is pH dependent. The release of nitric oxide proximate the glucose sensor can minimize the foreign body response, and thus minimize the concomitant disruption of the pH sensor due to local pH levels dropping to levels at which the sensor performance is degraded.

Various embodiments of the CGMs, and devices used in conjunction with the CGMs, are described in more detail below. Provided below are descriptions, which should be understood as representative, i.e., non-limiting, of representative examples of CGMs that emit light to inhibit, reduce, or prevent foreign body response.

In one embodiment, a percutaneous continuous glucose monitor is disclosed, which has been modified so that it can reduce foreign body response when the biosensor is injected into a user's skin.

The modifications include providing coatings that release exogenous nitric oxide, at appropriate local concentrations, and at sufficient duration, to result in anti-microbial effects decreased collagen production, increased vascularization around the implant, and other biological effects which reduce the foreign body response to the implanted sensor.

As discussed above, percutaneous CGMs include a biosensor which is injected beneath the skin, as well as a portion adhered to, and overlying the skin, that includes the electrical components that read the information on glucose levels, and send it, either over a wired connection, or a wireless connection, to a display. The components of a CGM, in addition to the biosensor, typically include a sensor array, an electronics module, a battery, and a telemetry/transmission portal. Means for wirelessly transmitting a signal from a CGM to a display are well known in the art, and are not further discussed herein.

### Coating the Glucose Sensor

In the percutaneous glucose monitors described herein, the glucose sensor is at least partially coated with a polymeric coating as described herein, which coating emits nitric oxide over time and helps inhibit the foreign body response to the implanted glucose sensor, as well as inhibit bacterial growth around the sensor.

The coating is a biocompatible coating. As used herein, sensor coatings are “biocompatible” if they optimize the clinical relevance of the sensor, avoid any negative local and systemic effects, and elicit the most appropriate local tissue response adjacent to the implant.

In addition to the biocompatible coating materials discussed above, representative biocompatible coatings for glucose sensors include polyurethane, Nafion, polyethylene glycol, silicone, zwitterionic polymers, polyesters, including polyhydroxyacids such as PLA, PGA, and PLGA, polyglycolic lactic acid (PGLA), polysulfone (PSU), gelatin, polyvinylpyrrolidone and copolymers thereof, which coatings can also include pendant SNO groups, embedded particles that include SNO-containing compounds, or, in some embodiments, small molecules comprising SNO groups blended into the polymeric coatings or the particles.

Where the coatings are biodegradable, the particles can emerge from the coatings over time as the coatings biodegrade, and the particles can release nitric oxide as the SNO-containing compounds come into contact with physiological fluids.

The coatings and/or embedded particles can also include one or more additional compounds that can inhibit the foreign body response. Representative examples include, for example, VEGF or compounds that promote VEGF, TNF-α and/or β inhibitors, including anti-TNF-α and/or β antibodies, halofunginone, anti-inflammatory compounds, such as dexamethasone and monobutyrin, and antimicrobial compounds.

Molecular interference with FBR can involve local immunosuppression with corticosteroids. Leukocyte and fibroblast activation can be dampened using anti-transforming growth factor-β antibody or halofunginone. Blood vessel development can be stimulated, to improve perfusion and performance of the bioactive implants, using pro-angiogenic vascular endothelial growth factor (VEGF), or other angiogenic compounds.

### Determination of Blood Glucose

In some embodiments, the biosensors use enzymatic approaches, such as GOx, and measure glucose oxidation, oxygen consumption and/or hydrogen peroxide formation as a way to determine blood glucose levels.

The biosensors have to be sensitive to the differences in tissue concentrations of oxygen and glucose, and various coating layers on the portion of the biosensor with the working electrode have been developed, which help to control the permeability of glucose and/or oxygen, so as to provide more reliable readings.

In some embodiments, biocompatible coatings are applied to the biosensor, and in some aspects of these embodiments, compounds which inhibit the foreign body response elute from these coatings.

These approaches are discussed in more detail below.

### Non-Enzymatic Electrochemical Glucose Sensors

In one embodiment, the CGM uses a non-enzymatic electrochemical glucose sensor rather than an enzymatic electrochemical glucose sensor. In one aspect of this embodiment, glucose is measured directly via direct electro-oxidation at high-surface area (i.e., porous) platinum electrodes, or through potentiometric detection dependent on pKa changes in a conducting polymer.

### Implantable Microdialysis Probes

In another embodiment, the CGM uses implantable microdialysis probes rather than an enzymatic electrochemical glucose sensor. Glucose in the interstitial fluid is measured by collecting dialysate. Microdialysis avoids direct implantation of a sensor, but the glucose measurement (i.e., recovery) has historically been erratic in vivo due to the foreign body response. The implantable microdialysis probes are improved by including the ability to emit nitric oxide over time, which minimizes the foreign body response, thus improving this technique.

### Percutaneous Needle-Type Microsensors

Percutaneous needle-type microsensors monitor hydrogen peroxide production amperometrically as a measure of the glucose concentration. The sensing cavity generally consists of a Pt—Ir wire working electrode coated with three functional layers: the inner selective layer, an enzyme layer, and the outer membrane. A silver/silver chloride (Ag/AgCl) wire is wrapped around the working electrode and serves as both a pseudo-reference and counter electrode. Though such sensors typically are characterized as having a shorter stabilization period (e.g., 2-4 h) compared to subcutaneous glucose sensors, the device penetrates through an opening in the dermis with concomitant infection risk. In some cases, frequent calibration (e.g., 2 times per day) can be required even after the stabilization period, due to changes in sensor response. Furthermore, the percutaneous nature of the device creates additional forces on the sensor, such as mechanical motion, that can lead to even greater inflammation. Minimization of the foreign body response, particularly the inflammatory response, can be useful in minimizing the need for calibration.

Due to the foreign body response, sensor lifetimes are typically on the order of 5-14 days, at which point the biosensor must be replaced. In this regard, patient compliance has remained poor. Using the nitric oxide-releasing polymers described herein, the sensor lifetimes can increase to as much as 8-31 days, for example, 14-31 days, or even more.

### Improvements in Selectivity Using Polymer Coatings/Permselective Films

Sensor accuracy is critical for compliance. However, a number of substances can interfere with the sensor response, as they are electroactive at the electrode potential used to oxidize hydrogen peroxide. Permselective membranes that operate via size exclusion and/or electrostatic repulsion mechanisms are often used to improve selectivity. The composition of such membranes must be accounted for when considering biocompatibility, as the polymer contacts tissue and may ultimately dictate the foreign body response (FBR).

The range of polymeric materials that have been evaluated as effective permselective films/coatings for the electrode include cellulose acetate, Nafion, electropolymerized films (e.g., polyphenol), and multilayer hybrids of these polymers.

Polyphenol permselective membranes are able to electropolymerize within an enzyme layer in a controllable manner, yielding a film with a thickness that is self-limiting (10-100 nm). As such, this simple approach is very attractive for reducing interferences. In some cases, such membranes also exclude surface-active macromolecules (i.e., proteins and platelets), protecting the surface from biofouling.

The use of mediators to shuttle electrons between the enzyme and the electrode can also minimize the impact of interfering species by lowering the working potential required to oxidize hydrogen peroxide. Representative redox mediators include ferrocene and osmium complexes, quinone compounds, metal phthalocyanines, carbon nanotubes, and conducting polymers.

### Oxygen Dependence

The electrochemical detection of hydrogen peroxide requires oxygen, a cofactor in the glucose oxidation (GOx) enzymatic reaction. Oxygen concentration in interstitial fluid is approximately ten times lower than the concentration of glucose in interstitial fluid, resulting in an “oxygen deficit” state. This is typically addressed by incorporating an outer diffusion-controlled membrane in the biosensor/electrode.

Low concentrations of oxygen lead to problems with the biosensor response to glucose (particularly dynamic range) due to stoichiometric imbalance between the two cofactors. Oxygen deficiency is mitigated by using polymeric membranes that reduce glucose diffusion or employ alternative electron mediators.

In some embodiments, membranes similar to those that exclude polar interferences are used to increase the ratio of oxygen/glucose permeability. Exemplary polymers include polyurethane, Nafion, silicone elastomer, polycarbonate, and layer-by-layer assembled polyelectrolytes.

Sensor performance issues due to changing oxygen levels are exacerbated due to the foreign body response, which results in local consumption of oxygen and glucose by inflammatory cells at the vicinity of the sensor. The oxygen diffusion to the sensor decays exponentially after sensor implantation due to changes in tissue permeability. Accordingly, the local release of nitric acid, proximate to the tissues surrounding the biosensor, decreases these performance issues, and allows the biosensor to be used for relatively longer periods of time before it is replaced.

### Stability and Degradation of Sensor Components

The failure of sensor components in vivo may be categorized as follows: 1) enzyme instability and leaching; 2) membrane degradation and delamination; and, 3) electrode passivation. Enzyme activity begins to decrease immediately both due to polymer entrapment and exposure to reactive oxidative species from sensor operation and the FBR (exposure to hydrogen peroxide and other reactive radicals).

Effective immobilization strategies can help ensure enzyme stability. Examples of such strategies include crosslinking the enzyme with bovine serum albumin (BSA) or with glutaraldehyde, entrapping the enzyme with or without covalent tethering, within polymeric matrices (e.g., hydrogels and sol-gel-derived materials), incorporating the enzyme into electropolymerized conducting polymers such as polypyrrole, and fixing the enzyme onto the electrode surface by electrostatic interactions generated by polyelectrolytes.

Nevertheless, even properly immobilized enzymes inherently lose activity over time due primarily to loss of non-covalently bonded FAD cofactor. However, deactivation by endogenously produced hydrogen peroxide from oxidase reactions also contributes to the loss of activity. Large glucose concentrations and requirements for adequate sensor signals imply high rates of peroxide production and concomitant enzyme deactivation.

Sensors typically include films or membranes used as sensing layers, barrier membranes, and/or biocompatible layers. These materials are prone to degradation from oxidative challenges, such as those caused by the foreign body response, as well as calcification and delamination. When a film becomes detached or degrades, sensor instability or failure automatically results. Electrode fouling (often called electrode passivation) is another cause of sensor instability, and occurs when diffusible small molecules come into contact with the surface of the electrode after penetration of the sensor membrane.

The use of coatings that provide local concentrations of nitric oxide, as described herein, can minimize the adverse effects caused by the foreign body response.

### In Vivo Calibration

Since the analytical performance of CGM sensors changes drastically upon implantation, it is necessary to define and assess sensor accuracy. Traditionally, the in vivo accuracy of such devices is evaluated using numerical point or rate accuracy. Current numerical and clinical accuracy criteria for CGM includes linear regressions and correlation coefficients; mean (or median) absolute and relative absolute difference (MAD and MARD); Clarke Error Grid Analysis (Clarke EGA); and, International Standard Organization (ISO) criteria.

Because CGM systems also provide information on glucose fluctuations, the continuous glucose-error grid (CG-EGA) has been introduced comprising of (1) a point-error grid analysis (P-EGA) that evaluates the sensor accuracy in terms of accurate blood glucose measurements; and, (2) a rate-error grid analysis (R-EGA) that assesses the prediction capability of the sensor.

These in vivo sensor evaluation methods require true blood glucose concentrations as determined using an external glucose measuring device (i.e., finger-prick glucose sensor). Reliable and reproducible procedures for calibration during in vivo monitoring are crucial to achieving accurate measurements.

CGM systems inherently estimate the blood glucose concentration by assuming the concentration of glucose in interstitial fluids will be substantially similar. This assumption is problematic because the ratio of blood/tissue glucose is not constant, but rather depends on the metabolic rates related to glucose and insulin physiology including glucose uptake by cells or from blood vessels, blood flow, and permeability of capillaries.

Glucose concentration discrepancies between blood and interstitial fluid are typically complex and vary based on time and concentration according to the physical state of the patient, including resting, hyperventilation, exercise, anoxia, and hypoxia. The lag time between blood and subcutaneous tissue glucose concentrations cause further inaccuracies for CGM devices. Under normal conditions (i.e., conditions in which glucose levels are not rapidly changing from activities such as exercising or eating), the physiological lag time between blood and interstitial fluid glucose ranges between 5 and 10 min. Longer, or unpredictable, lag times are created by physiological differences between individuals, intrinsic sensor lag time (typically on the order of seconds to a few minutes), and noise filtering. Lag is also created by tissue responses to the sensor such as electrode fouling, biofouling, and the foreign body encapsulation that impedes glucose diffusion to the sensor. Again, frequent calibrations using external glucose measuring devices are required to ensure CGM sensor accuracy.

Both “one-point” and “two-point” calibration procedures with blood glucose strips have been used to calibrate CGM sensors. The calibration process involves the conversion of the time-dependent current signal (i(t)) into an estimation of blood glucose concentration at a given time (CG(t)). Using the one-point calibration procedure, sensor sensitivity (S) is determined as the ratio between the current signal and the blood glucose concentration from a single blood glucose determination.

This approach is useful for highly selective sensors with near-zero output current at zero glucose concentration. A two-point calibration procedure is preferred when the sensor output observed in the absence of glucose (i0) is not negligible. Two-point calibrations involve an estimate of two parameters, S and i0, by determining blood glucose concentration and concomitant sensor current at two different time points. The glucose concentration is then estimated from the response current according to eq. 1. The two-point calibration curve is

$$CG ( t ) = ( i ( t ) - i 0 ) / S \qquad ( 1 )$$

and is actually less accurate due to error associated with electronic noise and the “true” finger prick blood glucose measurement (accepted as ±10% error on commercial glucose meters) that results in significant positive or negative measurement artifacts. A one-point calibration is thus considered more appropriate.

Even with an accurate calibration, repeated calibration is required as the sensor sensitivity changes over time due to physiological fluctuations and the foreign body response to the sensor. However, using the nitric oxide releasing polymers, as described herein, the number of required calibrations can be minimized, since inaccuracies caused by the foreign body response are diminished.

In another embodiment, a subcutaneous continuous glucose monitor is disclosed, which has been modified to reduce foreign body response when it is implanted.

### Long-Term Electrochemical Implantable (Subcutaneous) Glucose Sensors

In some embodiments, rather than using percutaneous injection of a biosensor, the CGM is a subcutaneous implant, such as an implantable microdialysis probe or a long-term electrochemical implantable glucose sensor. Whereas percutaneous glucose sensors are typically used for less than a month (in large part, due to the foreign body response), fully implantable (i.e., subcutaneous) glucose sensors can be used for significantly longer terms.

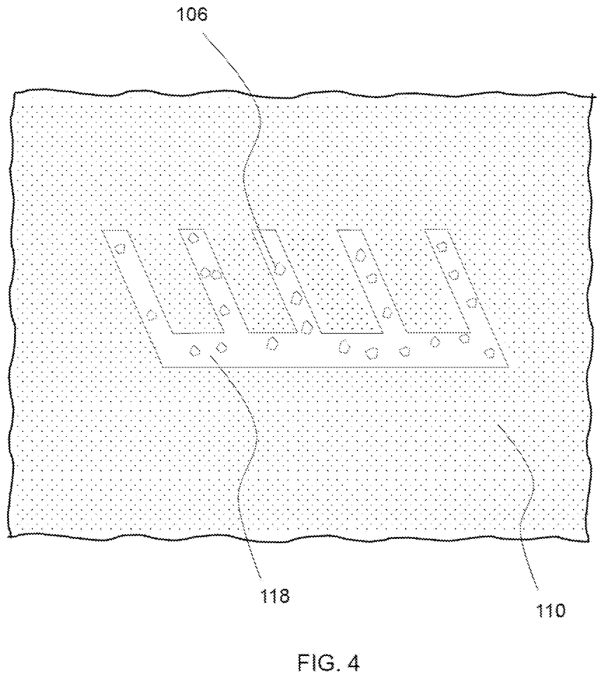

CGM devices with enzyme-immobilized amperometric biosensors can be implanted fully subcutaneously and used for extended periods (months to years). Subcutaneous glucose sensors typically include a disk-type sensor with a titanium housing and measure oxygen consumption (FIG. 4B). In one embodiment, the device detects glucose concentration using fluorescence or chemiluminescence, rather than GOx (glucose oxidation). One such device is the Eversense® device.

Fluorescent glucose biosensors typically measure the concentration of glucose by means of sensitive protein that relays the concentration by means of fluorescence. The majority of the fluorophores used for the sensors are small molecules, although some sensors have been made using quantum dots (QD) or fluorescent proteins.

Chemiluminescence, the generation of light by means of chemical reactions, is produced by some proteins, such as Aqueorin from symbiont in jellyfish and luciferase from symbiont in fireflies. These proteins have been used to make glucose sensors. For example, a Ggbp-split aqueorin-based sensor and a Ggbp-luciferase with Asp459Asn (Glc not Gal)-based sensor have been developed.

In one embodiment, the subcutaneous sensor uses differential electrochemical detection of oxygen via a two-step chemical reaction catalyzed by GOx and catalase. In some aspects of this embodiment, accurate glucose measurements can be carried out for more than one year by taking into account the difference in oxygen reduction at an electrode producing a glucose-modulated current and a reference electrode producing an oxygen-dependent current.

The size of the sensor is typically larger than percutaneous CGM systems (˜3 cm versus 3 microns) due to power (i.e., battery) requirements to support longer use. This is minimized, in some aspects of this embodiment, by using alternative means to provide long-term power supplies.

Such alternative means include those disclosed in Ben Amar et al., “Power Approaches for Implantable Medical Devices,” Sensors (Basel) 15(11):28889-28914 (2015).

In one aspect, energy is generated and harvested from potential sources surrounding the implants, for example, using biofuel cells that exploit glucose and oxygen, which are abundant in the blood to generate energy (see, for example, Wei and Liu, “Power sources and electrical recharging strategies for implantable medical devices,” Front. Energy Power Eng. China, 2:1-13 (2008).

In another aspect, body heat, or movements like breathing and motion, can be exploited to provide power to implanted medical devices (IMDs), and replace the need for traditional batteries. For example, thermoelectric generators can exploit the temperature difference between the inner parts and the skin (typically around 8° C.) to generate a few hundred microwatts of electricity. Piezoelectric generators can convert kinetic energy into electricity using piezoelectric materials. Electrostatic and electromagnetic mechanisms can be used to harvest energy using body motions.

In yet another embodiment, energy is supplied to IMDs using an external unit to either charge the battery, or to continuously power a “battery-less” implant. In various aspects of this embodiment, this can be accomplished optically, ultrasonically and/or electromagnetically. Optical-charging methods involve using a photovoltaic cell in the IMD which receives power from a light source which applies light whether using an LED, an OLED, or a laser, typically operating in the near-infrared or infrared range.

Inductive power transmission can also be used. This typically involves using a pair of antennas by which power is transferred through a mutual inductive coupling link. Those of skill in the art can readily determine an appropriate antenna design and orientation, working distance and frequency, as well as the designated power for the implanted device.

A limitation of conventional subcutaneous sensors is that the sensor response changes over time due to collagen encapsulation, variations in local microvascular perfusion, and limitations in oxygen availability.

Because implantation and subsequent replacement of the sensors requires surgery, in each instance followed by a long (˜2-3 week) stabilization period, it is desirable to minimize the number of times the devices need to be replaced, and, while in service, the number of times the device needs to be recalibrated.

To accomplish this, the implanted device can be coated with a coating as described herein, which provides a local concentration of nitric oxide around the implanted device. In some aspects of this embodiment, the coating comprises a polymer with pendant SNO groups, and in other aspects, the coating includes embedded particles, which particles comprise compounds, or particles, with pendant SNO groups.

### Methods for Measuring Blood Glucose Levels

Methods for using the devices to measure glucose levels, while minimizing foreign body response to the injected biosensors, are also disclosed. In one embodiment, the methods involve using the percutaneous glucose monitors, and in another embodiment, the methods involve using subcutaneous glucose monitors.

Where the device is a percutaneous glucose monitor, the approaches described herein allow the user to wear a continuous glucose monitor for a relatively longer period of time, in contrast to conventional percutaneous (continuous) glucose monitors, before having to remove and replace it due to inaccuracies in glucose readings resulting from the foreign body response.

Also disclosed are methods of inhibiting the foreign body response to an injected sensor, catheter, and

In some embodiments, the methods involve applying a percutaneous continuous glucose monitor, an insulin pump, or other device that includes an injected sensor, catheter, port, shunt, and the like, that is injected into the skin of a user, where the device comprises a coating comprising one or more compounds that produce nitric oxide.

In other embodiments, the embodiments involve implanting a subcutaneous continuous glucose monitor, an insulin pump, or other device that includes an injected sensor, catheter, or port, that is injected into the skin of a user, where the device comprises a coating comprising one or more compounds that produce nitric oxide.

The methods described herein can be used to treat, prevent, manage or lessen the severity of a foreign body response to an injected or implanted biosensor.

In some embodiments, the term “preventing” relates to preventing a foreign body response from occurring at all. In other embodiments, preventing relates to minimizing foreign body response, such that the biosensor does not lose sufficient sensitivity that would normally be seen as a result of foreign body response over a time period of around 21 days, or up to 31 days. In the context of the use of continuous glucose monitors, there are reasons other than the foreign body response that limit their useful lifetimes. For example, the continuous glucose monitors (CGMs) are adhered to the tissue, and the adhesive wears out over time. At around 30 days, whether or not the injected biosensor is still providing accurate readings, most users would seek to replace it, for example, due to loss of adhesion of the CGM to the skin.

The methods involve applying a CGM to the skin, which includes adhering the body of the CGM to the skin, while also injecting a biosensor into the skin, wherein the CGM comprises a coating that provides localized NO release to the tissue surrounding the biosensor. The NO provides antimicrobial effects, can reduce inflammation, and can increase vascularization.

Depending on the particular CGM, the device may need to be periodically calibrated. This is typically done by doing finger sticks, and measuring blood glucose levels.

The prevention, or minimization, of foreign body response means that the sensor retains its accuracy for a longer period of time, so the user can calibrate the CGM relatively less frequently than where the foreign body response is not prevented, or minimized.

### Tissue Engineering Scaffolds

In still other embodiments, the medical devices are scaffolds used in tissue engineering applications. A tissue engineering scaffold acts as an extracellular matrix that interacts with the cells prior to forming new tissues. The chemical and structural characteristics of scaffolds are major concerns in fabricating of ideal three-dimensional structure for tissue engineering applications. The polymer scaffolds used for tissue engineering ideally possess proper architecture and mechanical properties in addition to supporting cell adhesion, proliferation, and differentiation. The scaffolds are porous, and there is a tradeoff between mechanical strength and porosity, in that sufficient porosity should be present to allow for cellular infiltration, but not so much porosity that the mechanical strength of the scaffold is sacrificed.

Tissue scaffolds typically include cells that are intended to be grown on the scaffold, such as stem cells and other types of non-differentiated cells, and may include growth factors and other compounds that direct the propagation and differentiation of the cells. In one aspect of this embodiment, the stem cells proliferate in approximately the same timeframe as the scaffold degrades, thus forming a three-dimensional tissue matrix in approximately the same shape as the scaffold.

Because the tissue scaffolds are porous, and typically biodegrade as the cells embedded into the scaffolds propagate and/or differentiate, it can be desirable to not cover the porous scaffolds with a polymer that is not biodegradable, and/or is not porous. Accordingly, in one embodiment, the scaffold does not include a polymeric coating that includes a dye, pigment and/or light stabilizing compound. Rather, the polymers used to prepare the scaffolds, and/or particles or small molecules embedded within the polymers, include a dye, pigment and/or light stabilizing compound, so as to minimize decomposition of the NO-releasing functional groups in the polymers, particles and/or small molecules, as such functional groups are exposed to light.

The synthetic polymers poly(L-lactic acid) (PLLA), poly(glycolic acid) (PGA), poly(caprolactone) (PCL) and poly(lactic-co-glycolic) acid (PLGA) are commonly used to form three dimensional structures in the form of scaffolds, by themselves, or in combination with natural polymers, which can help improve hydrophilicity, cell attachment, and biodegradability. These polymers can be prepared using thiolactic acid, or another hydroxy acid with a thiol side chain, so as to incorporate nitrosothiol groups in the final tissue scaffold when the thiol groups are converted to nitrosothiol groups. Where the thiol groups can interfere with the polymerization chemistry, or would be converted to other functional groups, and not be available for later nitrosation to form nitrosothiol groups, the thiol groups can be protected during the polymerization process, and deprotected afterwards. Protecting groups for thiols are well known to those of skill in the art.

In one aspect of this embodiment, the scaffold is formed from polymers that include pendant NO-releasing groups, such as diazeniumdiolate and/or nitrosothiol (SNO) groups. In another aspect of this embodiment, the scaffold is formed from polymers comprising embedded particles, which particles comprise one or more compounds. The polymers, and/or embedded particles, can be the same polymers and particles discussed above with respect to the NO-releasing coatings.

### Stitches/Surgical Staples

In another embodiment, the devices are resorbable or non-resorbable stiches or surgical staples, which can release nitric oxide into the wound site to inhibit infection and promote wound healing. Degradable, or absorbable sutures can be broken down by the human body without the need for external removal, and can be characterized by their loss of 50% or more of their tensile strength within four weeks after implementation. Degradable sutures can be made from both natural and synthetic polymers. Sterility is important during both the manufacture and usage of these devices to minimize the event of infection as a result of the introduction of foreign materials into the body.

These devices can be used, for example, to close a wound or surgical incision, while also releasing nitric oxide over time, which can aid in wound healing, increasing vascularization, minimize scarring, and reduce instances of infection. In this embodiment, dyes, pigments and/or light stabilizing compounds can be mixed into the polymers to provide light stabilization to the devices, such that the premature release of NO, such as might occur during long term storage in packaging that permits the sutures to be exposed to light, is minimized.

Degradable sutures are commonly prepared from PLA, PGA, PLGA, and polydioxanone (PDS), a synthetic homopolymer, prepared through the polymerization of the monomer paradioxanone, which has the following formula:

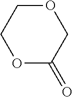

These types of sutures are often prepared by melt extrusion of the polymeric materials into the form of a monofilament. The melt extrusion process might decompose certain NO-releasing functional groups, so in those embodiments where the NO-releasing functional groups would be significantly decomposed during the melt extrusion process, the NO-functional groups can be formed after the sutures are prepared.

As with other degradable materials formed from PLA, PGA, PLGA, and the like, thiol-containing monomers, such as thiolactic acid, cysteine, and the like, can be blended in with the monomers used to prepare the degradable materials, and thus provide a biodegradable polymeric material with pendant thiol groups. These thiol groups can then be converted to NO-releasing nitrosothiol groups, in some embodiments, after the sutures are fabricated.

In some embodiments, the degradable polymers themselves do not comprise NO-releasing functional groups, but comprise embedded small molecules or particles that comprise NO-releasing functional groups.

### Monoliths/Films/Tapes

In still other embodiments, a “monolith,” or a tape or film, which incorporates NO-releasing groups, on small molecules and/or polymers, is physically or chemically attached to a medical device, such as an implant. In some aspects of these embodiments, the implant/monolith or implant/tape composite is coated with a layer that comprises a dye, pigment, or light stabilizing compound, and in other aspects, the monolith or tape further comprises a dye, pigment or light stabilizing compound.

In some aspects of this embodiment, the tapes/films coat all or a portion of medical devices selected from the group consisting of arterial stents, guide wires, catheters, trocars, needles, bone anchors, bone screws, protective platings, hip or joint replacements, electrical leads, biosensors, probes, sutures, surgical drapes, wound dressings, and bandages.

The tape or film can be physically or chemically attached to a medical device, such as an implant. Where the tape or film is chemically attached, it preferably comprises a biocompatible, and preferably biodegradable, adhesive. Such adhesives are well-known to those of skill in the art. One and two-part epoxy and silicone biocompatible adhesives can be used, as can various light-cured materials, epoxy-polyurethane blends, and cyanoacrylates. In one embodiment, the adhesive is a biocompatible and biodegradable polyurethane adhesive. In another embodiment, the adhesive is a poly (glycerol sebacate acrylate) (PGSA).

### Surgical Glues/Tissue Adhesives

An ideal tissue adhesive, especially for pulmonary, cardiovascular and/or gastrointestinal applications, ideally has all or most of the following properties:
(1) low viscosity or liquid-like properties prior to curing to enable easy application to a desired area,
(2) minimum washout by body fluids and activation only when desired to facilitate delivery and repositioning of implanted devices during minimally invasive procedures,
(3) significant adhesive strength, especially in the presence of blood and/or other body fluids,
(4) ability to resist the mechanical loads from adhesion to highly mobile tissue (e.g. contractions of the heart, or pulsations in large vessels),
(5) ability to form a hemostatic seal,
(6) minimal inflammatory response, and
(7) biodegradability.

In addition to these properties, it can be advantageous for the surgical glue to release nitric oxide over time, as this can minimize microbial contamination that often accompanies surgery, promote wound healing, increase vascularization, and minimize scar formation. The surgical glues/tissue adhesives described herein provide this nitric oxide release.

Many surgical glues in use today include one or more of (meth)acrylate functional groups, or cyanoacrylates, or a combination of albumin and glutaraldehyde, or include poly(ethylene glycol) (PEG) blocks, or include polyurethane, or are composed of fibrin.

Cyanoacrylates belong to a class of monomers consisting of the alkyl esters of 2-cyanoacrylic acid. To date, methyl, ethyl, n-butyl, isobutyl, isohexyl and octyl cyanoacrylates have been used. Butyl-2-cyanoacrylate adhesives include Indermil® (Covidien), Histoacryl® and Histoacryl® Blue (TissueSeal), and LiquiBand® (Advanced Medical Solutions).

Octyl-2-cyanoacrylate adhesives include Dermabond® (Ethicon), SurgiSeal™ (Adhezion Biomedical), LiquiBand® Flex (Advanced Medical Solutions), and OctylSeal (Medline Industries).

Cyanoacrylates offer tensile strength similar to that of absorbable sutures for closure of skin wounds, and are capable of adhering to most tissue surfaces, but are not suggested for use in high-tension areas, across joints, on mucosal surfaces, at mucocutaneous junctions, or areas of dense hair growth.

Artiss (Baxter) is a fibrin product, approved for adhering skin grafts to wounded skin caused by burns and for tissue flaps during facial rhytidectomy surgery. This fibrin product is formed from pooled human plasma.

Where medical grade cyanoacrylate (CA) or fibrin sealants are often used, they may not have all or most of the desired properties for surgical adhesives. However, particles or small molecules that release nitric oxide can be blended with these surgical glues, and at least they can have the beneficial properties associated with nitric oxide release.

BioGlue® (CryoLife) is a surgical adhesive approved for use in vascular sealing of large blood vessels in conjunction with sutures for the purpose of hemostasis, and to assist in the repair of aortic dissection to provide a stronger vessel wall after vascular surgery. BioGlue is a mixture of a purified bovine serum albumin (BSA) and glutaraldehyde, which polymerizes in situ at the application site within 30 seconds with full strength achieved in 2 minutes.

TissuGlu® (Cohera Medical Inc) is used in abdominal tissue bonding to help reduce fluid accumulation under skin. TissuGlu® is applied to the underlying abdominal layer to reapproximate the skin flap with the muscle layer, and aids in the prevention of seroma, a pocket of clear serous fluid, under the skin after abdominoplasty (tummy tuck). This product includes a hyperbranched polyurethane with isocyanate end groups, and lysine. The amine groups in the lysine crosslink with the isocyanate groups, with adhesive crosslinking taking place within 25 minutes.

PEG-based sealants include FocalSeal® (Genzyme Biosurgery), Progel™ (Neomend), Duraseal™ and DuraSeal™ Xact (Covidien), Coseal® (Baxter), and ReSure Sealant (Ocular Therapeutix, Inc.) are commercially available PEG-based sealants currently approved by the FDA for clinical uses. While they are all categorized as PEG-based, differences exist in the polymers used and their indicated uses.

Focalseal® (Genzyme Biosurgery, Inc. Cambridge, MA) is a polyethylene glycol-based synthetic hydrogel, which is a block copolymer including one or more polyethylene glycol blocks and one or more PLGA blocks, which also include carbonate linkages, and which includes (meth)acrylate end caps. The adhesive minimizes tissue adhesion (and thus minimizes scarring) by virtue of the polyethylene glycol block, and is degradable by virtue of the PLGA block. It can also be adhered to skin using the (meth)acrylate terminal end groups, which can be reacted with functional groups on the tissue surface by applying light and an amine (which generates the free radicals used to cure the (meth)acrylate groups) to the skin surface.

Focalseal® is a PEG-co-trimethylene carbonate-co-lactide with acrylated end groups, and eosin Y is added as a component to react with light after the adhesive is applied, to produce the free radicals that polymerize the polymer in situ. Focalseal® is FDA-approved as a sealant to limit airleak following pulmonary resection, and has also been used as a hemostatic adjunct to prevent anastomotic bleeding and to seal other types of closure such as the dura, pancreatic stump, and open wounds. The sealant has two components, a primer and a sealant, and is applied in two steps, after which (meth)acrylate end-capping groups on the polymer are then polymerized using visible light, typically a blue-green light. The sealant is degraded by hydrolysis of the biodegradable block. The sealant is flexible, in part by virtue of the carbonate linkages, and nontoxic.

ProGel™ includes human serum albumin solution (HSA) and di-PEG-succinimidyl succinate, which crosslink with each other, and which are administered using a dual syringe to avoid having the components mix before application, so they can be cured in situ by crosslinking upon application.

DuraSeal™ includes tetra-PEG-succinimidyl ester and trilysine amine, which are administered using a dual syringe and crosslink when applied. DuraSeal™ is used as an adjunct to sutured dural repair during cranial surgery to provide watertight closure.

Coseal®, which includes a tetra-PEG-succinimidyl ester and is tetra-thiol-derivatized, is used to manage anastomotic bleeding during aortic reconstruction after graft implantation and to stop bleeding from anastomotic suture holes.

Improvements to these types of adhesives are disclosed herein. In one embodiment, the surgical glue is a block copolymer comprising one or more polyalkylene glycol blocks, such as polyethylene glycol blocks, and one or more degradable blocks.

In some embodiments, the degradable blocks are formed from any suitable combination of degradable monomeric units, such as lactic acid, glycolic acid, hydroxybutyric acid, caprolactone, carbonates, and the like, and in some embodiments, are a peptide such as trilysine or other short chain (i.e., less than 25 monomeric units) peptide that comprises more than two lysine monomeric units, or a protein, such as albumin.

In some embodiments, the surgical glue also comprises a vinyl group (such as a (meth)acrylate group) that can be polymerized via free radical polymerization. In other embodiments, the surgical glue is a two or more component system, where one component includes a functional group that can crosslink with a functional group on another component.

In one embodiment, a polyalkylene glycol, such as polyethylene glycol, block comprises a functional group that crosslinks with a different functional group on a degradable block. Those of skill in the art understand what functional groups are capable of crosslinking with other functional groups under physiological conditions.

In one aspect of this embodiment, the degradable blocks comprise one or more monomeric units that comprise pendant thiol or amine groups, which can be modified to form nitrosothiol, diazeniumdiolate, or other NO-releasing groups before the glue is applied. In this embodiment, it is important that the nitrosothiol, diazeniumdiolate, or other NO-releasing functional groups do not interfere with the crosslinking chemistry. In another embodiment, the surgical glue is blended with particles or small molecules that comprise NO-releasing functional groups. Combinations of these approaches can be used.

Other surgical glues which are cured by the free-radical polymerizatyion of vinyl groups, such as (meth)acrylate groups, include those disclosed in U.S. Pat. No. 8,143,042 to Bettinger et al. The '042 patent discloses biodegradable elastomers prepared by crosslinking a pre-polymer containing crosslinkable functional groups, such as acrylate groups. In some embodiments, the pre-polymer can have a molecular weight of between about 300 Daltons and 75,000 Daltons, and have varying degrees of (meth)acrylation.

The primary mechanism of adhesion of the polymer disclosed in the '042 patent, and many other surgical adhesives known in the art, is chemical interactions between functional groups (e.g. free hydroxyl groups) on the polymer and the tissue to which it is applied. Thus, in some embodiments, the surgical glue need not include two or more components that crosslink with each other, so long as the surgical glue includes one or more functional groups that crosslink with groups found on the tissue surface to be adhered.

The elastomers can be modified to include monomer units with pendant thiol or amine groups, which can be converted to nitrosothiol groups, diazeniumdiolate groups, or other NO-releasing groups, and thus provide NO release after the glue is applied, and the surgical glue is exposed to physiological fluids. Alternatively, the surgical glue can be blended with particles or small molecules that comprise NO-releasing functional groups.

Similarly, Mandavi, et al., PNAS, 2008, 2307-2312 describes nanopatterned elastomeric PGSA polymer with a thin layer of oxidized dextran with aldehyde functionalities (DXTA) to increase adhesion strength of the adhesive by promoting covalent cross-linking between terminal aldehyde group in DXTA with amine groups in proteins of tissue. This adhesion mechanism is based essentially on covalent bonding between the radicals generated during the curing process and functional groups on the tissue surface. While this type of reactive chemistry can potentially promote undesirable immune reactions, such as local inflammation, the local release of nitric oxide can minimize the immune reactions, leading to a more stable surgical adhesion.

U.S. Pat. No. 9,724,447 also discloses surgical glues, and these glues comprise pre-polymers with flow characteristics such that they can be applied through a syringe or catheter but are sufficiently viscous to remain in place at the site of application and not run off the tissue. The pre-polymers are also sufficiently hydrophobic to resist washout by bodily fluids, and are stable in bodily fluids. That is, the pre-polymers do not spontaneously crosslink in bodily fluids absent the presence of an intentionally applied stimulus to initiate crosslinking. Upon crosslinking, the adhesive exhibits significant adhesive strength in the presence of blood and other bodily fluids. The adhesive is sufficiently elastic that it is able to resist movement of the underlying tissue, and can a hemostatic, biodegradable and biocompatible seal.

The pre-polymers are of the formula (-A-B-)ₙ, wherein A is derived from a substituted or unsubstituted polyol moiety, B is derived from a substituted or unsubstituted diacid, and n represents an integer greater than 1. The pre-polymer comprises a plurality of polymeric backbones which are activated with functional groups comprising substituted or unsubstituted vinyl groups, including (meth)acrylate groups, crosslinkable by exposure to light, heat, or chemical (free-radical) initiators. The pre-polymers have a weight average molecular weight of between about 1,000 and less than 20,000 Daltons.

In one embodiment, not disclosed in the '447 patent, at least a portion the diacid and/or the diol monomers used to prepare the surgical glue comprise a pendant thiol or amine group, which is converted to a nitrosothiol or diazenium diolate group, or other NO-releasing group, before the glue is applied. In another embodiment, the surgical glue is blended with particles or small molecules that comprise NO-releasing functional groups. In either of these embodiments, it can be preferred that the surgical glue be cured using a free radical initiating agent, rather than light or heat, as light and heat can at least partially decompose the NO-releasing groups, and reduce the NO payload of the surgical glue.

### Subcutaneous Implants

In other embodiments, the medical devices are subcutaneous implants that comprise a coating that includes biocompatible, and, in some cases, biodegradable polymers, and optionally includes embedded particles. Representative subcutaneous implants include artificial joints, pacemakers, subcutaneous glucose monitors, stents, insulin infusion sets, shunts, such as hydrocephiletic shunts, reconstructive cosmetic implants, including breast, calf, and butt implants. With respect to stents, the release of nitric oxide from the coatings on the stents can minimize restenosis.

### Medical Implants

A medical implant is a medical device manufactured to replace a missing biological structure, support a damaged biological structure, or enhance an existing biological structure. Medical implants are man-made devices, in contrast to transplants, which are transplanted biomedical tissue. The surface of implants that contact the body might be made of a biomedical material such as titanium, silicone, apatite, and/or plastic, such as high density polyethylene (HDPE) or ultra-high density polyethylene UHDPE), depending on the device.

In some cases, such as artificial pacemakers, implantable cardioverter-defibrillators and cochlear implants, the implants contain electronics. Some implants, such as subcutaneous drug delivery devices, are bioactive. Representative subcutaneous drug delivery devices include implantable pills and drug-eluting stents. Orthopedic implants can be used to repair fractures or replace missing bone and/or cartilage. Certain implants assist with the function of an organ or an organ system. Examples include coronary, gastrointestinal, respiratory, and urological implants. Sensory and neurological implants can also be treated using the tapes and monoliths described herein. A coronary stent, such as a drug-eluting stent, is another common item implanted in humans. Orthopedic implants are used to repair fractures, such as those in the radius and ulna.

Additional details regarding the various types of implants are provided below.

### Sensory and Neurological Implants

Sensory and neurological implants are used for disorders affecting the major senses and the brain, as well as other neurological disorders. They are predominately used to treat conditions such as cataract, glaucoma, keratoconus, and other visual impairments; otosclerosis and other hearing loss issues, as well as middle ear diseases such as otitis media; and neurological diseases such as epilepsy, Parkinson's disease, and treatment-resistant depression. Examples include the intraocular lens, intrastromal corneal ring segment, cochlear implant, tympanostomy tube, and neurostimulator.

### Cardiovascular Implants

Cardiovascular medical devices are implanted in cases where the heart, its valves, and the rest of the circulatory system is in disorder. They are used to treat conditions such as heart failure, cardiac arrhythmia, ventricular tachycardia, valvular heart disease, angina pectoris, and atherosclerosis. Examples include the artificial heart, artificial heart valve, implantable cardioverter-defibrillator, cardiac pacemaker, and coronary stent.

### Orthopedic Implants

Orthopedic implants help alleviate issues with the bones and joints of the body. They are used to treat bone fractures, osteoarthritis, scoliosis, spinal stenosis, and chronic pain. Examples include a wide variety of pins, rods, screws, and plates used to anchor fractured bones while they heal.

Representative orthopedic implants include the Austin-Moore prosthesis for fracture of the neck of the femur, Baksi's prosthesis for elbow replacement, Charnley prosthesis for total hip replacement, Condylar blade plate for condylar fractures of femur, Ender's nail for fixing intertrochanteric fracture, Grosse-Kempf nail for tibial or femoral shaft fracture, Hansson pin (or LIH for Lars Ingvar Hansson), a hook-pin used for fractures of the femoral neck, Harrington rod for fixation of the spine, Hartshill rectangle for fixation of the spine, Insall Burstein prosthesis, for total knee replacement, Wohns inter-spinous implant and implantation instrument intended to be implanted between two adjacent dorsal spines, Kirschner wire for fixation of small bones, Kuntscher nail for fracture of the shaft of the femur, Luque rod for fixation of the spine, Moore's pin for fracture of the neck of the femur, Neer's prosthesis for shoulder replacement, Rush nail for diaphyseal fractures of a long bone, Smith Peterson nail for fracture of the neck of the femur, Smith Peterson nail with McLaughlin's plate for intertrochanteric fractures, Seidel nail for fracture of the shaft of humerus, Souter's prosthesis for elbow replacement, Steffee plate for fixation of the spine, Steinmann pin for skeletal traction, Swanson prosthesis for the replacement of joints of the fingers, Talwalkar nail for fracture of the radius and ulna, and Thompson prosthesis for fracture of the neck of femur.

### Electrical Implants

Electrical implants can be used, for example, to relieve pain and suffering from rheumatoid arthritis or chronic back or neck pain. In one embodiment, an electrical implant is embedded in the neck of patients with rheumatoid arthritis, and the implant sends electrical signals to electrodes in the vagus nerve.

Neurostimulation is approved as a treatment for chronic lower back pain (CLBP), and an implant, such as ReActiv8 (Mainstay Medical) can be used to treat CLBP. These types of implants work by sending electrical signals that stimulate dormant nerve tissue within the multifidus.

The deep multifidus muscle (specifically, the section in the lower back) is one of the most important stabilizers of the lumbar spine-critical for walking, sitting, and especially bending. When this muscle atrophies from lack of use or degrades from overuse/injury, people commonly experience impaired motor control in the lower back. An implant can be used to treat multifidus muscle dysfunction, by using electrical stimulation of a nerve (neurostimulation) to induce contraction in the lower back muscle, correcting the muscle weakness that causes lower back pain.

The deep multifidus muscle (specifically, the section in the lower back) is one of the most important stabilizers of the lumbar spine-critical for walking, sitting, and especially bending. When this muscle atrophies from lack of use or degrades from overuse/injury, people commonly experience impaired motor control in the lower back.

This impaired control is one of the key underlying causes of CLBP. Accordingly, implants intended to treat CLBP can function by reviving the contracting abilities of the multifidus, which can re-enable control of the lumber spine. An implanted pulse generator can provide electrical stimulation to the dorsal ramus nerve, the nerve that runs through the multifidus. This stimulation can induce repetitive contractions of the multifidus muscle, and thus address the cause of CLBP.

### Contraceptive Implants

Contraceptive implants are primarily used to prevent unintended pregnancy and treat conditions such as non-pathological forms of menorrhagia. Examples include copper- and hormone-based intrauterine devices.

A contraceptive implant is a type of hormonal birth control, which typically progestin hormone into the body to prevent pregnancy. In one embodiment, the implant is a very small plastic rod about the size of a matchstick, which is inserted it into the upper arm, under the skin. An intrauterine device is another type of contraceptive implant.

Nitric oxide reduces sperm motility, possibly by inhibiting cellular respiration independent of an elevation of intracellular cGMP. Nitric oxide elaborated in the female or male genital tract in vivo can adversely influence sperm function and fertility. Weinberg J B, Doty E, Bonaventura J, Haney AF, “Nitric oxide inhibition of human sperm motility,” Fertil Steril. 1995 August; 64(2):408-13 (1995). Accordingly, a diaphragm that releases nitric oxide can further reduce the likelihood of pregnancy, by not only physically blocking sperm from reaching the egg, but also by inhibiting sperm motility. A conventional diaphragm can be modified by adhering a tape or monolith to it, or by spraying one or more sides of the diaphragm with a sprayable formulation, before it is inserted.

### Cosmetic Implants

Cosmetic implants, including prosthetics, attempt to bring some portion of the body back to an acceptable aesthetic norm. They are used as a follow-up to mastectomy due to breast cancer, for correcting some forms of disfigurement, and modifying aspects of the body (as in buttock augmentation and chin augmentation). Examples include breast, calf, chin and buttocks implants, nose prostheses, ocular prostheses, and testicular prostheses.

### Cardiac Implants

Cardiac implants include pacemakers, implantable cardioverter-defibrillator, and stents, including drug-loaded stents.

A cardiac pacemaker generates electrical impulses, delivered by electrodes, to cause the heart muscle chambers (the upper, or atria and/or the lower, or ventricles) to contract and therefore pump blood. Pacemakers replace and/or regulate the function of the electrical conduction system of the heart, by maintaining an adequate heart rate. In some embodiments, the pacemaker is externally programmable, and allows a cardiologist to select the optimal pacing modes for individual patients. In some embodiments, the pacemaker is a demand pacemaker, in which the stimulation of the heart is based on the dynamic demand of the circulatory system. One type of pacemaker is a defibrillator, which combines pacemaker and defibrillator functions in a single implantable device. Another type is a biventricular pacemakers, which includes multiple electrodes stimulating differing positions within the lower heart chambers to improve synchronization of the ventricles, the lower chambers of the heart.

An implantable cardioverter-defibrillator (ICD) or automated implantable cardioverter defibrillator (AICD) is a device implantable inside the body, able to perform cardioversion, defibrillation, and (in modern versions) pacing of the heart. The device is therefore capable of correcting most life-threatening cardiac arrhythmias. The ICD is the first-line treatment and prophylactic therapy for patients at risk for sudden cardiac death due to ventricular fibrillation and ventricular tachycardia. Current devices can be programmed to detect abnormal heart rhythms and deliver therapy via programmable anti-tachycardia pacing in addition to low-energy and high-energy shocks.

### Implants That Stimulate Other Organs and Organ Systems

Other types of organ dysfunction can occur in the systems of the body, including the gastrointestinal, respiratory, and urological systems. Implants are used in those and other locations to treat conditions such as gastroesophageal reflux disease, gastroparesis, respiratory failure, sleep apnea, urinary and fecal incontinence, and erectile dysfunction.

Examples include insulin pumps, the LINX, implantable gastric stimulator, diaphragmatic/phrenic nerve stimulator, neurostimulators, surgical mesh, artificial urinary sphincter and penile implants.

### Porous Implants

In some embodiments, the implants are porous. Porosity in implants serves two primary purposes. The elastic modulus of the implant is decreased, allowing the implant to better match the elastic modulus of the bone. The elastic modulus of cortical bone (˜18 MPa) is significantly lower than typical solid titanium or steel implants (110 MPa and 210 MPa, respectively), causing the implant take up a disproportionate amount of the load applied to the appendage, leading to an effect called stress shielding. This undesired effect can be minimized by using a porous implant.

Porosity also enables osteoblastic cells to grow into the pores of implants. Cells can span gaps of smaller than 75 microns and grow into pores larger than 200 microns. Bone ingrowth is a favorable effect, as it anchors the cells into the implant, increasing the strength of the bone-implant interface. More load is transferred from the implant to the bone, reducing stress shielding effects. The density of the bone around the implant is likely to be higher due to the increased load applied to the bone. Bone ingrowth reduces the likelihood of the implant loosening over time because stress shielding, and corresponding bone resorption, is minimized. In embodiments where it is desired to have osteoblasts penetrate into the implant, it can be desirable for the implant, or at least the surface of the implant, to have a degree of porosity greater than 40%, to facilitate sufficient anchoring of the osteoblasts.

In some embodiments, to avoid issues associated with foreign body response, all or a portion of the pores can be filled with a degradable material that releases NO over time, which helps to minimize the foreign body response. As the material degrades, osteoblasts can fill in the pores, particularly where the material used to fill the pores is seeded with osteoblasts, and, optionally, fibroblast growth factors can be included, as these can help control osteoblast differentiation (P. J. Marie, “Fibroblast growth factor signaling controlling osteoblast differentiation,” Gene, Volume 316, pp. 23-32 (2003)).

### Percutaneous Implants

In some embodiments, the medical devices are percutaneous implants. Representative percutaneous implants include percutaneous glucose monitors, catheters/ports, including urinary catheters and venous ports/catheters for chemotherapy (e.g port-a-cath), as well as stomal ports, fluid-draining devices (drains), drug delivery devices, blood-sampling devices, and percutaneously implanted neurostimulator electrode arrays.

Catheters, fluid-draining devices (i.e., drains), drug delivery devices and blood sampling devices can be modified by adhering a tape or monolith, or sprayed with a sprayable formulation as described herein, where the tape, monolith, or sprayed-on formulation releases nitric oxide upon implantation. The release of nitric oxide can inhibit bacterial growth in and around the devices, and inhibit the foreign body response to the devices. By way of example, urinary catheters can cause urinary tract infections, and the release of nitric oxide from the catheters can minimize the likelihood of infection.

Fluid-draining device (e.g., drains) can be used, for example, to drain ascites fluid or fluid that builds up around a patient's heart, or fluid that builds up around a surgical site, and the release of nitric oxide can minimize microbial contamination and promote wound healing.

Drug delivery and blood sampling devices typically include a tube that is inserted into a patient for delivering a drug over an extended period of time or taking repeated blood samples. Examples include ports, such as chest ports. The tissue surrounding these ports can be subject to infection and/or the foreign body response, which can be minimized using the tapes, monoliths, or sprayable formulations described herein.

### Methods of Treatment

Methods of treatment using the devices described herein are also disclosed. The medical devices prepared according to the methods described herein are used to deliver NO to a treatment site in an individual or animal. A “treatment site” includes a site in the body of an individual or animal in which a desirable therapeutic effect can be achieved by contacting the site with NO. An “individual” refers to a human and an animal includes veterinary animals such as dogs, cats and the like and farm animals such as horses, cows, pigs and the like.

Treatment sites include, for example, sites within the body that develop a foreign body response to an implanted medical device. Where the medical device is a continuous glucose monitor, the foreign body response may cause the glucose sensor to become fouled, which results in the need for the continuous glucose monitor to be replaced.

Where the medical device is a stent, restenosis, injury or thrombosis can result due to trauma caused by contacting the site with a synthetic material or a medical device. For example, restenosis can develop in blood vessels which have undergone coronary procedures or peripheral procedures with PTCA balloon catheters (e.g. percutaneous transluminal angioplasty). Restenosis is the development of scar tissue from about three to six months after the procedure and results in narrowing of the blood vessel. NO reduces restenosis by inhibiting platelet deposition and smooth muscle proliferation. NO also inhibits thrombosis by inhibiting platelets and can limit injury by serving as an anti-inflammatory agent.

Treatment sites can also develop at non-vascular sites, for example at sites where a useful therapeutic effect can be achieved by reducing an inflammatory response. Examples include the airway, the gastrointestinal tract, bladder, uterine and corpus cavernosum. Thus, the compositions, methods and devices described herein can be used to treat respiratory disorders, gastrointestinal disorders, urological dysfunction, impotence, uterine dysfunction and premature labor. NO delivery at a treatment site can also result in smooth muscle relaxation to facilitate insertion of a medical device, for example in procedures such as bronchoscopy, endoscopy, laparoscopy and cystoscopy. Delivery of NO can also be used to prevent cerebral vasospasms post hemorrhage and to treat bladder irritability, urethral strictures and biliary spasms.

The method of delivering NO to a treatment site in an individual or animal comprises implanting a medical device coated with a polymer, sprayed with a composition, or to which a tape or monolith is applied, as described herein, at the treatment site. NO can be delivered to bodily fluids, for example blood, by contacting the bodily fluid with a medical device coated with a polymer of the present invention. A preferred polymer is an S-nitrosylated polymer, as defined above. Examples of treatment sites in an individual or animal, medical devices suitable for implementation at the treatment sites and medical devices suitable for contacting bodily fluids such as blood are described in the paragraphs hereinabove.

“Implanting a medical device at a treatment site” refers to bringing the medical device into actual physical contact with the treatment site or, in the alternative, bringing the medical device into close enough proximity to the treatment site so that NO released from the medical device comes into physical contact with the treatment site. A bodily fluid is contacted with a medical device coated with a polymer of the present invention when, for example, the bodily fluid is temporarily removed from the body for treatment by the medical device, and the polymer coating is an interface between the bodily fluid and the medical device. Examples include the removal of blood for dialysis or by heart lung machines.

Methods of monitoring glucose levels using a percutaneous glucose monitor, with a sensor coated with an NO-releasing coating, are also disclosed. In some embodiments, the coating further comprises a dye, pigment and/or light stabilizing compound that minimizes premature degradation of the NO-releasing compounds in the coating, or a second coating comprising a dye, pigment and/or light stabilizing compound overlies the coating. This also applies to the sprays, tapes and monoliths described herein.

### Methods of Minimizing Foreign Body Response Using the Implanted Medical Devices

Methods of minimizing foreign body response to implanted medical devices, such as percutaneous or subcutaneous implants, by coating NO-releasing medical devices as described herein, spraying an NO-releasing composition onto the devices, applying an NO-releasing tape to the devices, and/or applying an NO-releasing monolith to the devices, are also disclosed. The medical devices all comprise a polymer, particle, or small molecule that comprises NO-releasing functional groups that release nitric oxide when exposed to physiological fluids. In some embodiments, the coatings, sprays, tapes and/or monoliths also include a pigment, dye or light stabilizing compound that minimizes decomposition of the NO-releasing functional groups when exposed to light.

Implantation of these devices therefore releases nitric oxide, which can reduce the foreign body response to the implanted devices, relative to devices that do not include a coating that includes, or are not formed from materials that release nitric oxide over time.

The methods described herein can be used to treat, prevent, manage or lessen the severity of a foreign body response to an injected or implanted medical device. In some embodiments, the term “preventing” relates to preventing a foreign body response from occurring at all.

The present invention will be better understood with reference to the following non-limiting examples.

### Example 1: Medical Device Comprising Particles that Release NO and Block Light

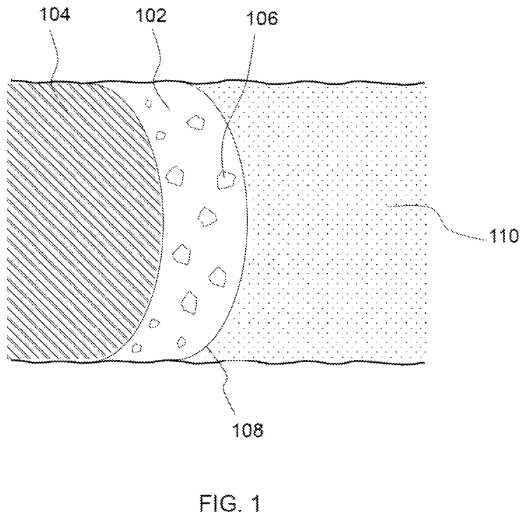

FIG. 1 shows a representative medical device (104), such as a stent, port, sensor, and the like, which comes into contact with human tissue (110) at an interface (108). The medical device (104) comprises an NO-releasing coating, tape or monolith (102), which coating, tape or monolith comprises NO-releasing biodegradable particles (106).

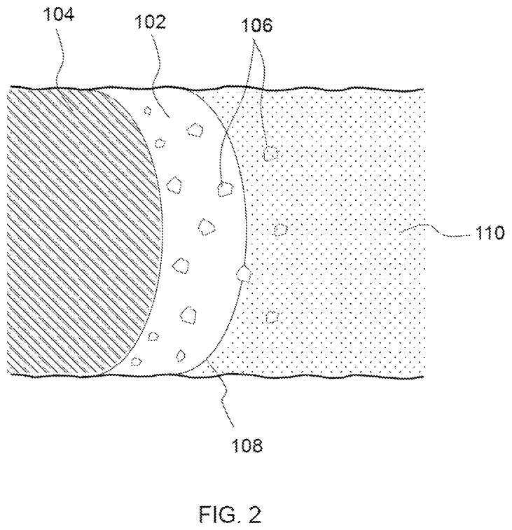

FIG. 2 is the same as FIG. 1, except that it shows the condition where some of the particles (106) are diffused out of the coating, and left within the host tissue. In this state, they may continue to release any remaining NO payload or simply degrade via normal metabolic pathways.

### Example 2: Surgical Glue/Sealant

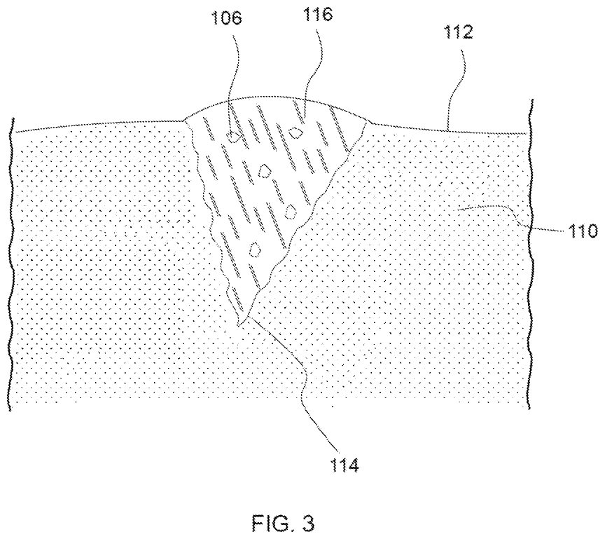

FIG. 3 is a drawing of a surgical glue (116) comprising NO-releasing biodegradable particles (106). The glue is shown placed within a wound site (114) of a host tissue (110). The glue is applied over the skin surface (112) where the wound has separated the skin, and is applied to the full depth of the wound. The release of nitric oxide promotes wound healing, and helps to reduce scar formation. As such, the surgical glues described herein accelerate the healing process.

### Example 3: Tissue Scaffold

FIG. 4 is a drawing of a tissue scaffold (118) on a substrate, such as human tissue, when implanted, or a petri dish, plate, and the like, when not implanted (110) comprising NO-releasing biodegradable particles (106). The scaffold can be prepared from the same material as the particle, or can be prepared from a significantly different material, depending on the requirements for each specific application. The NO promotes tissue ingrowth and vascularization, and reduces scar tissue formation.

### Example 4: NO Releasing Percutaneous Glucose Sensor

### Particle Fabrication

A polycaprolactone solution was prepared and used to fabricate biodegradable particles; a waterborne hydrophilic polyurethane dispersion was used as the base coating solution. A polycaprolactone (PC) (4.50 g, ALDRICH cat. Num. 440744) was placed into a glass bottle (250 mL) into which tetrahydrofuran (180.0 ml, ALDRICH cat. Num. 401757-1L) was also placed. The vial was sonicated in a heated bath (about 40° C.) until the polymer completely dissolved (4-6 hours). To this solution, thiolactic acid (TLA) (0.50 g, ALDRICH cat. Num. T31003-100G) was added to create a mixture now having about 3% overall solids by weight. The solution was homogenized using a laboratory vortex mixer (30 seconds). The solution was then loaded into a Buchi Nano Spray Dryer B-90 HP and used to prepare 200 nm dried particles. The resulting biodegradable particles consisted of about 10% TLA by weight.

### Particle Nitrosation

The TLA-loaded PC particles (100 mg) were placed into a glass scintillation vial (20 mL). Particles were suspended in −20° C. MeOH (5.0 mL). Then, an HCl solution (5M, 2.0 mL) was added to the vial. In a second vial (20 mL), NaNO₂ (0.100 g) was dissolved in an EDTA solution (500 μM, 2.0 mL). This solution was then combined with the first vial and the reaction allowed to proceed for 2 hours at 0° C. in the dark. The crude reaction mixture was placed in a foil-covered conical tube (50 mL) along with −20° C. methanol (30 mL). This tube was mixed, allowed to sit for 2 minutes. Then, the tube was placed in a centrifuge (4500 rpm, 10 min, 4° C.) so as to drop the particles to the bottom of the tube, after which the supernatant was discarded. The particles were resuspended in cold methanol 3 times in order to wash them in this manner. After the final wash, the particles were dried in a vacuum chamber (1 hour at −30 in Hg). In this way, the TLA embedded PC particles were loaded with NO.

### CGM Coating

The NO loaded PC particles (100 mg) were placed in a scintillation vial (20 mL). To this vial was also added a waterborne polyurethane dispersion (1.2 mL, Baymedix CD104, COVESTRO, Pittsburgh, PA) and suspended in the dispersion using a laboratory vortex mixer (10 seconds). The CGM sensor was coated using Chemat DipMaster 50 Dip Coater (Northridge, CA). The sensor was dipped in the solution (3 coats, 5-minute dry time between coatings) resulting in a final coating thickness of 20-40 μm.

Persons of skill in the art will recognize medical devices that may be as yet unknown to them (currently existing or developed in the future) and that would be capable of delivering nitric oxide to the tissue surrounding implanted medical devices, such as an injected biosensor, for example, including biosensors other than those which operate via a glucose oxidase mechanism, and devices that are applied to tissue rather than being implanted. All such devices are within the scope of devices that can be used in carrying out methods described herein.

### Example 5: Medical Device Comprising a Tape Comprising Particles that Release NO

### Polymer Synthesis

A polycaprolactone solution was prepared and used to fabricate biodegradable particles; a waterborne hydrophilic polyurethane dispersion was used as the base coating solution. A polycaprolactone (PC) (4.50 g, ALDRICH cat. Num. 440744) was placed into a glass bottle (250 mL) into which tetrahydrofuran (180.0 ml, ALDRICH cat. Num. 401757-1L) was also placed. The vial was sonicated in a heated bath (about 40° C.) until the polymer completely dissolved (4-6 hours). To this solution, thiolactic acid (TLA) (0.50 g, ALDRICH cat. Num. T31003-100G) was added to create a mixture now having about 3% overall solids by weight. The solution was homogenized using a laboratory vortex mixer (30 seconds). The solution was then loaded into a Buchi Nano Spray Dryer B-90 HP and used to prepare 200 nm dried particles. The resulting biodegradable particles consisted of about 10% TLA by weight.

### Particle Nitrosation

The TLA-loaded PC particles (100 mg) were placed into a glass scintillation vial (20 mL). Particles were suspended in −20° C. MeOH (5.0 mL). Then, an HCl solution (5M, 2.0 mL) was added to the vial. In a second vial (20 mL), NaNO₂ (0.100 g) was dissolved in an EDTA solution (500 μM, 2.0 mL). This solution was then combined with the first vial and the reaction allowed to proceed for 2 hours at 0° C. in the dark. The crude reaction mixture was placed in a foil-covered conical tube (50 mL) along with −20° C. methanol (30 mL). This tube was mixed, allowed to sit for 2 minutes. Then, the tube was placed in a centrifuge (4500 rpm, 10 min, 4° C.) so as to drop the particles to the bottom of the tube, after which the supernatant was discarded. The particles were resuspended in cold methanol 3 times in order to wash them in this manner. After the final wash, the particles were dried in a vacuum chamber (1 hour at −30 inHg). In this way, the TLA embedded PC particles were loaded with NO.

### Tape Preparation

The NO loaded PC particles (100 mg) were placed in a scintillation vial (20 mL). To this vial was also added a waterborne polyurethane dispersion (1.2 mL, Baymedix CD104, COVESTRO, Pittsburgh, PA) and suspended in the dispersion using a laboratory vortex mixer (10 seconds). The mixture was coated onto a siliconized release liner using a doctor blade film coater. The film was dried in a vacuum chamber (1 hour at −30 in Hg) resulting in a polyurethane film (0.30 mm thick) having NO loaded PC particles embedded therein.

The contents of all documents referred to herein are hereby incorporated by reference for all purposes.

Persons of skill in the art will recognize devices that may be as yet unknown to them (currently existing or developed in the future) and that would be capable of delivering light to the tissue surrounding the injected biosensor, for example, in conjunction with biosensors other than those which operate via a glucose oxidase mechanism, and all such devices are within the scope of devices that can be used in carrying out methods described herein.

1. A medical device comprising a polymer coating that comprises:
a) one or more polymers that
i) comprise NO-releasing functional groups, or
ii) are blended with particles or small molecules that include such NO-releasing functional groups.

2. The medical device of claim 1, wherein at least one of the polymers is biocompatible and/or biodegradable.

3. The medical device of claim 1, wherein at least one of the polymers is biocompatible and non-biodegradable.

4. The medical device of claim 1, wherein the polymer is hydrophobic.

5. The medical device of claim 1, wherein the polymer is formed from a mixture that comprises lactic acid, glycolic acid, carbonate, amino acid, or caprolactone monomers, or mixtures thereof, and also includes one or more thiol-containing monomers, wherein all or a portion of the thiol groups on the thiol-containing monomers are converted to nitrosothiols.

6. (canceled)

7. The medical device of claim 1, wherein the device is a percutaneous or subcutaneous implant.

8. (canceled)

9. The medical device of claim 7, wherein the percutaneous implant is a percutaneous glucose monitor that comprises an implantable glucose sensor,
wherein the implantable glucose sensor comprises
i) a first portion that is an active sensing region, and
ii) a second portion that operatively connects the active sensing region to the rest of the percutaneous glucose monitor, and allows the active sensing region to penetrate the skin of the user to a desired depth to measure glucose levels, and an NO-releasing polymeric coating on all or part of the second portion of the implantable glucose sensor.

10-13. (canceled)

14. The percutaneous glucose monitor of claim 9, wherein the polymeric coating comprises a porous polymer located on all or part of the first and second portion of the glucose sensor, wherein:
a) the porous polymer used to form the coating comprises one or more NO-releasing functional groups,
b) the porous polymer used to form the coating comprises one or more embedded particles, wherein the particles comprise a polymer comprising one or more NO-releasing functional groups,
c) the porous polymer used to form the coating is blended with one or more small molecules, which small molecules have a molecular weight less than 1,000 and comprise one or more NO-releasing functional groups, or
d) combinations thereof.

15-20. (canceled)

21. The medical device of claim 12, wherein the hydrophobic polymer is a hydrophobic polyurethane.

22. The medical device of claim 12, wherein the particles are formed of a polymer comprising lactic acid, glycolic acid, and thiolactic acid monomers, wherein the thiol functional group on one or more of the thiolactic acid monomers is converted to a nitrosothiol group.

23-25. (canceled)

26. The medical device of claim 7, wherein the device is an artificial joint, a stent, a shunt, a port, an insulin infusion set, a catheter, a pacemaker, or a reconstructive cosmetic implant.

27-28. (canceled)

29. The medical device of claim 7, wherein the polymeric coating comprises a hydrophobic polymer.

30-31. (canceled)

32. The medical device of claim 29, wherein the hydrophobic polymer is a hydrophobic polyurethane.

33. The medical device of claim 29, wherein the particles are formed of a polymer comprising lactic acid, glycolic acid, and thiolactic acid monomers, wherein the thiol functional group on one or more of the thiolactic acid monomers is converted to a nitrosothiol group.

34-38. (canceled)

39. A medical device comprising a polymer that comprises one or more NO-releasing functional groups, and/or comprises embedded particles and/or small molecules that include such NO-releasing functional groups.

40. The medical device of claim 39, wherein the device is a resorbable or non-resorbable stitch, a staple, a monolith or a tape which incorporates NO-releasing groups.

41. The medical device of claim 40, wherein the monolith or tape is physically or chemically attached to a medical device.

42. The medical device of claim 39, wherein the device is a resorbable or non-resorbable tissue scaffold.

43. The medical device of claim 39, wherein the polymer is a hydrophobic polymer.

44-46. (canceled)

47. The medical device of claim 39, wherein the particles are formed of a polymer comprising lactic acid, glycolic acid, and thiolactic acid monomers, wherein the thiol functional group on one or more of the thiolactic acid monomers is converted to a nitrosothiol group.

48-50. (canceled)

51. A method for delivering nitric oxide from the surface of a biomedical implant, comprising implanting an implant of claim 1 into a patient, and following implantation, allowing the NO-releasing groups on the polymer surface to be exposed to biological fluids, which cause the groups to release nitric oxide.

52. A method for measuring blood glucose concentration, comprising:
a) implanting the implantable glucose sensor of the percutaneous glucose monitor of claim 9 into a patient, and
b) measuring blood glucose concentrations using the percutaneous glucose monitor, while also
allowing the NO-releasing groups on the polymer coating to be exposed to biological fluids, which cause the groups to release nitric oxide, wherein nitric oxide release occurs for a period of at least three days.

53-54. (canceled)

55. A medical device comprising an adhered tape or monolith, or a film formed by application of a sprayable polymeric formulation, wherein the tape, monolith, or film comprises one or more biodegradable polymers that:
i) comprise NO-releasing functional groups, or
ii) are blended with particles or small molecules that include such NO-releasing functional groups,
wherein the medical device is a sensory implant, neurological implant, cardiac implant, orthopedic implant, electrical implant, contraceptive implant, or cosmetic implant.

56-59. (canceled)

60. The medical device of claim 55, wherein the polymer in the tape, monolith, or applied film is formed from a mixture that comprises lactic acid, glycolic acid, carbonate, amino acid, or caprolactone monomers, or mixtures thereof, and also includes one or more thiol-containing monomers, wherein all or a portion of the thiol groups on the thiol-containing monomers are converted to nitrosothiols.

61. (canceled)

62. The medical device of claim 55, wherein the device is a percutaneous or subcutaneous implant.

63-67. (canceled)

68. The medical device of claim 55, wherein the device is an artificial joint, a stent, a shunt, a port, an insulin infusion set, a catheter, a pacemaker, or a reconstructive cosmetic implant.

69-70. (canceled)

71. The medical device of claim 55, wherein the monolith or tape is physically or chemically attached to a medical device.

72. The medical device of claim 55, wherein all or a portion of the device is porous.

73. The medical device of claim 72, wherein the sprayable formulation is applied to, and at least partially fills, one or more of the pores.

74. A method for delivering nitric oxide from the surface of a biomedical implant, comprising implanting an implant of claim 55 into a patient, and following implantation, allowing the NO-releasing groups on the polymer surface to be exposed to biological fluids, which cause the groups to release nitric oxide.

75. A method for modifying a medical device such that it releases nitric oxide, comprising:
a) adhering a tape or monolith to a medical device, wherein the tape or monolith comprises a polymer that comprises one or more pendant NO-releasing functional groups, and/or incorporates embedded particles or small molecules that comprise one or more pendant NO-releasing functional groups, or
b) spraying a medical device with a sprayable composition comprising
i) a polymer that comprises one or more pendant NO-releasing functional groups,
ii) a polymer, and particles and/or small molecules that comprise one or more pendant NO-releasing functional groups;
to a medical device prior to implantation of the device in or application of the device on as patient, and
c) implanting the device in a patient, or applying the device on a patient.
